(19) United States
(12) Patent Application Publication
(10) Pub. No.: US 2010/0098633 A1
(43) Pub. Date: Apr. 22, 2010

(54) SELECTIVE SEPRASE INHIBITORS

(75) Inventors: Craig ZIMMERMAN, Topsfield, MA (US); John W. Babich, Cambridge, MA (US); John Joyal, Melrose, MA (US); John Marquis, Nashua, NH (US); Jian-cheng Wang, Revere, MA (US)

Correspondence Address: FOLEY AND LARDNER LLP;SUITE 500, 3000 K STREET NW, WASHINGTON, DC, 20007, US

(73) Assignee: Molecular Insight Pharmaceuticals, Inc.

(21) Appl. No.: 12/566,324

(22) Filed: Sep. 24, 2009

Related U.S. Application Data
(60) Provisional application No. 61/100,178, filed on Sep. 25, 2008.

(51) Int. Cl. A61K 51/04; A61K 31/69; A61K 31/555; A61K 31/40; C07F 13/00; A61P 35/00

(52) U.S. Cl. 424/1.85; 424/1.65; 514/64; 514/184; 514/423; 544/225; 546/12; 548/402

(57) ABSTRACT

Novel radiopharmaceuticals that are useful in diagnostic imaging and therapeutic treatment of disease characterized by overexpression of seprase include complexes that contains a proline moiety and a radionuclide adapted for radioimaging and/or radiotherapy:

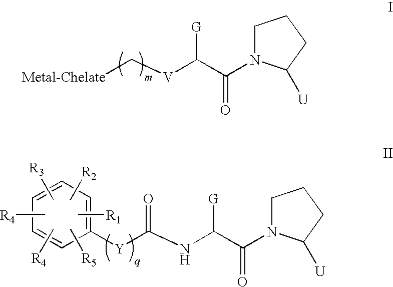

### CROSS-REFERENCE TO RELATED APPLICATIONS

This application claims the benefit of U.S. Provisional Patent Application No. 61/100,178, filed Sep. 25, 2008, the disclosure of which is incorporated herein by reference in it's entirety.

### FIELD

This invention relates in general to small molecule inhibitors of seprase that can be used as therapeutic agents through inhibition of seprase's enzymatic activity, or as radiopharmaceuticals that bind to seprase and therefore enable imaging of tissues that express seprase or for delivering radiotherapy to tumor tissues that express seprase.

### BACKGROUND

Seprase, also known as fibroblast activation protein alpha (FAP-α), is a transmembrane serine peptidase that belongs to the prolyl peptidase family. The prolyl peptidase family includes serine proteases that cleave peptide substrates after a proline residue. Seprase is expressed in epithelial cancers and has been implicated in extracellular matrix remodeling, tumor growth, and metastasis.

The prolyl peptidase family includes enzymes such as, but not limited to, dipeptidyl peptidase-IV (DPP-IV), DPP-VII, DPP-VIII, DPP-IX, prolyl oligopeptidase (POP), acylpeptide hydrolase and prolyl carboxypeptidase. These enzymes differ in structure at the N terminus, but are related in that each has a C-terminal αβ-hydrolase domain that contains the catalytic Ser, Asp, and H is residues. Similar to seprase, human DPP-IV is expressed constitutively on brush border membranes of intestine and kidney epithelial cells and is transiently expressed in activated T-cells and migratory endothelial cells.

The expression of distinct proteins on the surface of tumor cells offers the opportunity to diagnose and characterize disease by probing the phenotypic identity and biochemical composition and activity of the tumor. Radioactive molecules that selectively bind to specific tumor cell surface proteins allow for the use of noninvasive imaging techniques, such as molecular imaging or nuclear medicine, for detecting the presence and quantity of tumor associated proteins. Such methods may provide vital information related to the diagnosis and extent of disease, prognosis and therapeutic management options. For example, therapy may be realized through the use of radiopharmaceuticals that are not only capable of imaging disease, but also are capable of delivering a therapeutic radionuclide to the diseased tissue. The expression of seprase on tumors makes it an attractive target to exploit for noninvasive imaging as well as targeted radiotherapy.

Furthermore, since seprase has both dipeptidyl peptidase and endopeptidase activity, and DPP-IV exhibits only dipeptidyl peptidase activity, selective seprase inhibitors would be useful to reduce unwanted side effects.

### SUMMARY

Small molecule inhibitors of seprase are provided for use as therapeutic medicines or as radiopharmaceuticals useful in diagnostic imaging and in the therapeutic treatment of diseases characterized by overexpression of seprase. The radiopharmaceuticals include complexes or compounds that contain a functionalized proline moiety which is capable of selectively inhibiting seprase, and a radionuclide adapted for radioimaging and/or radiotherapy.

In one aspect, a complex of Formula I, its enantiomer, stereoisomer, racemate or pharmaceutically acceptable salt is provided:

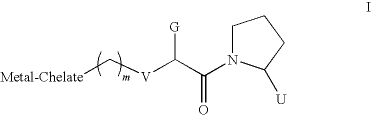

where:
U is —B(OH)₂, —CN, —CO₂H, or —P(O)(OPh)₂;
G is H, alkyl, substituted alkyl, carboxyalkyl, heteroalkyl, aryl, heteroaryl, heterocycle, or arylalkyl;
V is a bond, O, S, NH, (CH₂—CH₂—X)ₙ, or a group of formula

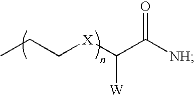

X is O, S, CH₂, or NR;
R is H, Me or CH₂CO₂H;
W is H or NHR′;
R′ is hydrogen, acetyl, t-butyloxycarbonyl (Boc), 9H-fluoren-9-ylmethoxycarbonyl (Fmoc), trifluoroacetyl, benzoyl, benzyloxycarbonyl (Cbz) or substituted benzoyl;
n is an integer ranging from 0 to 6;
m is an integer ranging from 0 to 6;
Metal represents a metallic moiety including a radionuclide; and
Chelate represents a chelating moiety that coordinates with said radionuclide.

In another aspect, a compound of general Formula II, its enantiomer, stereoisomer, racemate or pharmaceutically acceptable salt is provided:

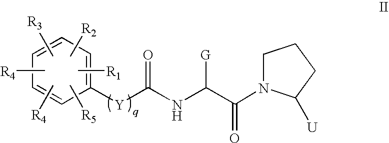

where:
U is —B(OH)₂, —CN, —CO₂H, or —P(O)(OPh)₂;
G is H, alkyl, substituted alkyl, carboxyalkyl, heteroalkyl, aryl, heteroaryl, heterocycle, or arylalkyl;
Y is a bond, —O—, —CH₂—, —OCH₂—, NR, —NR—CH₂, or CH₂—NR—;
R is H, Me or CH₂CO₂H;
q is an integer ranging from 0 to 24; and
R₁, R₂, R₃, R₄ and R₅ are independently hydrogen, halogen, cyano, carboxyl, alkyl, alkylamino, alkoxy, or substituted or unsubstituted amino; with the proviso that at least one of R₁, R₂, R₃, R₄ and R₅ is a halogen (including radiohalogen).

In another aspect, a method of imaging tissue of a mammal which expresses seprase is provided which includes administering to the mammal an effective amount of a radiolabeled compound or complex that selectively inhibits seprase or binds to the enzymatic domain of seprase. In one embodiment, the radiolabeled complex includes a metal radionuclide-containing chelate derivative of a seprase inhibitor. In another embodiment, the radiolabeled compound includes a radioactive halogenated derivative of a seprase inhibitor. In another embodiment, an effective amount of a complex or compound of Formula I and II, its enantiomer, stereoisomer, racemate or pharmaceutically acceptable salt is administered to the mammal.

In a further aspect, a method of treating a mammal suffering a disease which is characterized by overexpression of seprase, is provided. The method includes administering to the mammal a therapeutically effective amount of a radiolabeled seprase inhibitor, such as a radionuclide-containing chelate derivative, or a radioactive halogen derivative. In some embodiments, the method includes administering to a mammal a complex or compound of Formula I or II, its enantiomer, stereoisomer, racemate or pharmaceutically acceptable salt.

In still another aspect, a kit is provided including the subject complexes or compounds and a pharmaceutically acceptable carrier, and optionally instructions for their use. Uses for such kits include therapeutic management and medical imaging applications.

### BRIEF DESCRIPTION OF THE DRAWINGS

FIG. 1 is a graphical representation of the data presented in Table 1: Percent of Control versus Concentration of Inhibitor, for several compounds presented in the examples.

FIG. 2 presents radiochromatogram of the HPLC purified 1-131 labelled Compound 1024 in comparison to non-radiolabelled Compound 1024 as an identity standard, according to one embodiment.

FIG. 3 shows stability of radiolabeled Compound 1024 after 24 hours (bottom radiochromatogram) in comparison to after one hour (top), according to one embodiment.

FIG. 4 shows stability of Compound 1109, at 5 hours, according to one embodiment.

FIG. 5 is a graphical representation of seprase cell based enzyme assay with Compound 1024. Cells were incubated for 15 min. +/−25 μM, according to one embodiment.

FIG. 6 is a graph of the tissue biodistribution of Compound 1014/1109 in normal mice, expressed as % ID/g±(SEM), according to one embodiment.

FIG. 7 is a graph of the tissue biodistribution of Compound 1018/1110 in normal mice, expressed as % ID/g±(SEM), according to one embodiment.

FIG. 8 is a graph of the tissue biodistribution of 1-131 labeled Compound 1024 in FaDu Xenograft mice, expressed as % ID/g±(SEM), according to one embodiment.

FIG. 9 is a graph of the tissue biodistribution of 1-123 labeled Compound 1024 in H22(+) Xenograft mice after 1 hour, with or without blocking, expressed as % ID/g (SEM), according to one embodiment.

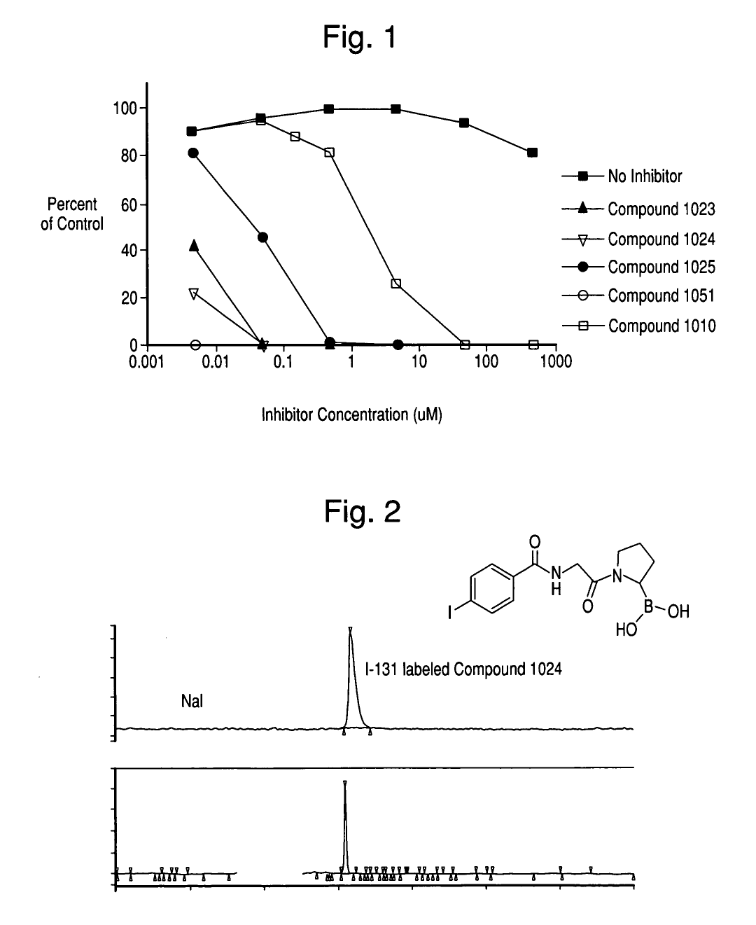

### DETAILED DESCRIPTION OF PREFERRED EMBODIMENTS

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As used herein, the following definitions of terms shall apply unless otherwise indicated.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

“Complex” refers to a compound formed by the union of one or more electron-rich and electron-poor molecules or atoms capable of independent existence with one or more electronically poor molecules or atoms, each of which is also capable of independent existence.

“Ligand” refers to a species that interacts in some fashion with another species. In one example, a ligand may be a Lewis base that is capable of forming a coordinate bond with a Lewis Acid. In other examples, a ligand is a species, often organic, that forms a coordinate bond with a metal ion. Ligands, when coordinated to a metal ion, may have a variety of binding modes know to those of skill in the art, which include, for example, terminal (i.e., bound to a single metal ion) and bridging (i.e., one atom of the Lewis base bound to more than one metal ion).

“Chelate” or “chelating agent” refers to a molecule, often an organic one, and often a Lewis base, having two or more unshared electron pairs available for donation to a metal ion. The metal ion is usually coordinated by two or more electron pairs to the chelating agent. The terms, “bidentate chelating agent”, “tridentate chelating agent”, and “tetradentate chelating agent” refer to chelating agents having, respectively, two, three, and four electron pairs readily available for simultaneous donation to a metal ion coordinated by the chelating agent. Usually, the electron pairs of a chelating agent forms coordinate bonds with a single metal ion; however, in certain examples, a chelating agent may form coordinate bonds with more than one metal ion, with a variety of binding modes being possible.

“Radionuclide” refers to molecule that is capable of generating a detectable image that can be detected either by the naked eye or using an appropriate instrument, e.g. positron emission tomography (PET) and single photon emission computed tomography (SPECT). Radionuclides useful within the present disclosure include penetrating photon emitters including gamma emitters and X-ray emitters. These rays accompany nuclear transformation such as electron capture, beta emission and isomeric transition. Radionuclides useful include those with photons between 80 and 400 keV and positron producers, 511 keV annihilation photons and acceptable radiation doses due to absorbed photons, particles and half life. Radionuclides include radioactive isotopes of an element. Examples of radionuclides include ¹²³I, ¹²⁵I, ^{99m}TC, ¹⁸F, ⁶²Cu, ¹¹¹In, ¹³¹I, ¹⁸⁶Re, ⁹⁰Y, ²¹²Bi, ²¹¹At, ⁸⁹Sr, ¹⁶⁶Ho, ¹⁵³Sm, ⁶⁷Cu, ⁶⁴Cu, ¹⁰⁰Pd, ²¹²Pb, ¹⁰⁹Pd, ⁶⁷Ga, ⁶⁸Ga, ⁹⁴Tc, ¹⁰⁵Rh, ⁹⁵Ru, ¹⁷⁷Lu, ¹⁷⁰Lu, ¹¹C, and ⁷⁶Br. “Radiohalogen,” as used herein, refers to those radionuclides that are also halogens (i.e. F, Br, I, or At).

“Coordination” refers to an interaction in which one multi-electron pair donor coordinatively bonds (is “coordinated”) to one metal ion.

“Tether” refers to a chemical linking moiety between a metal ion center and another chemical moiety.

“Lewis base” and “Lewis basic” are art-recognized and generally refer to a chemical moiety capable of donating a pair of electrons under certain reaction conditions. It may be possible to characterize a Lewis base as donating a single electron in certain complexes, depending on the identity of the Lewis base and the metal ion, but for most purposes, however, a Lewis base is best understood as a two electron donor. Examples of Lewis basic moieties include uncharged compounds such as alcohols, thiols, and amines, and charged moieties such as alkoxides, thiolates, carbanions, and a variety of other organic anions. In certain examples, a Lewis base may consist of a single atom, such as oxide (O₂⁻). In certain, less common circumstances, a Lewis base or ligand may be positively charged. A Lewis base, when coordinated to a metal ion, is often referred to as a ligand.

In general, “substituted” refers to a group, as defined below (e.g., an alkyl or aryl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group will be substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, alkynoxy, aryloxy, aralkyloxy, heterocyclyloxy, and heterocyclylalkoxy groups; carbonyls(oxo); carboxyls; esters; urethanes; oximes; hydroxylamines; alkoxyamines; aralkoxyamines; thiols; sulfides; sulfoxides; sulfones; sulfonyls; sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

Alkyl groups include straight chain and branched alkyl groups having from 1 to 20 carbon atoms or, in some embodiments, from 1 to 12, 1 to 8, 1 to 6, or 1 to 4 carbon atoms. Alkyl groups further include cycloalkyl groups. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above. Where the term haloalkyl is used, the alkyl group is substituted with one or more halogen atoms.

Cycloalkyl groups are cyclic alkyl groups such as, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Cycloalkyl groups further include mono-, bicyclic and polycyclic ring systems, such as, for example bridged cycloalkyl groups as described below, and fused rings, such as, but not limited to, decalinyl, and the like. In some embodiments, polycyclic cycloalkyl groups have three rings. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups, which may be substituted with substituents such as those listed above.

Alkenyl groups include straight and branched chain and cycloalkyl groups as defined above, except that at least one double bond exists between two carbon atoms. Thus, alkenyl groups have from 2 to about 20 carbon atoms, and typically from 2 to 12 carbons or, in some embodiments, from 2 to 8, 2 to 6, or 2 to 4 carbon atoms. In some embodiments, alkenyl groups include cycloalkenyl groups having from 4 to 20 carbon atoms, 5 to 20 carbon atoms, 5 to 10 carbon atoms, or even 5, 6, 7, or 8 carbon atoms. Examples include, but are not limited to vinyl, allyl, CH═CH(CH₃), CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl, among others. Representative substituted alkenyl groups may be mono-substituted or substituted more than once, such as, but not limited to, mono-, di- or tri-substituted with substituents such as those listed above.

Alkynyl groups include straight and branched chain alkyl groups, except that at least one triple bond exists between two carbon atoms. Thus, alkynyl groups have from 2 to about 20 carbon atoms, and typically from 2 to 12 carbons or, in some embodiments, from 2 to 8, 2 to 6, or 2 to 4 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃), among others. Representative substituted alkynyl groups may be mono-substituted or substituted more than once, such as, but not limited to, mono-, di- or tri-substituted with substituents such as those listed above.

Aryl, or arene, groups are cyclic aromatic hydrocarbons that do not contain heteroatoms. Aryl groups include monocyclic, bicyclic and polycyclic ring systems. Thus, aryl groups include, but are not limited to, phenyl, azulenyl, heptalenyl, biphenylenyl, indacenyl, fluorenyl, phenanthrenyl, triphenylenyl, pyrenyl, naphthacenyl, chrysenyl, biphenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl or halo groups, bonded to one of the ring members. Rather, groups such as tolyl are referred to as substituted aryl groups. Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with substituents such as those listed above.

“Heteroalkyl” refers to alkyl groups containing one or more heteroatoms (e.g., N, O, S, or the like) as part of the hydrocarbyl groups, and having in the range of 1 up to about 10 carbon atoms. Exemplary heteroalkyl groups include hydroxyalkyl, aminoalkyl, mercaptoalkyl, e.g., hydroxymethyl, aminobutyl, 4-guanidinylbutyl, 3-indolylmethyl, mercaptomethyl, and the like.

“Carboxyalkyl” refers to alkyl groups containing one or more carboxylic acids, e.g. carboxymethyl, carboxyethyl and the like.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Amino acid” refers to all compounds, whether natural, unnatural or synthetic, which include both an amino functionality and an acid functionality, including amino acid analogs and derivatives.

“Carboxy” or “carboxyl” refers to —COOH or salts thereof.

“Amino” refers to the group —NH₂. “Cyano” refers to the group —CN. “Carbonyl” refers to the divalent group —C(O)— which is equivalent to —C(═O)—. “Nitro” refers to the group —NO₂. “Oxo” refers to the atom (═O). “Sulfonyl” refers to the divalent group —S(O)₂—. “Thiol” refers to the group —SH. “Thiocarbonyl” refers to the divalent group —C(S)— which is equivalent to —C(═S)—. “Hydroxy” or “hydroxyl” refers to the group —OH.

“Heteroatom” refers to an atom of any element other than carbon or hydrogen. Exemplary heteroatoms are boron, nitrogen, oxygen, phosphorus, sulfur and selenium.

“Halogen” or “halo group” refers to —F, —Cl, —Br or —I, including its radioactive isotopes such as ¹²³I, ¹²⁵I, ¹³¹I, ¹⁸F or ⁷⁶Br.

“Haloalkyl” refers to alkyl groups substituted with 1 to 5, 1 to 3, or 1 to 2 halo groups, wherein alkyl and halo are as defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Acyl includes the “acetyl” group CH₃C(O)—.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, cycloalkenyl-C(O)O—, substituted cycloalkenyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyl” refers to the group —C(O)NR¹⁰R¹¹ independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonyl” refers to the group —C(S)NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyl” refers to the group —SO₂NR¹⁰ R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Arylalkyl” refers to alkyl groups containing one or more aryl groups, e.g. arylmethyl, arylethyl and the like.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl, thiadiazolyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Preferred heteroaryls include pyridinyl, pyrrolyl, indolyl, thiophenyl, thiadiazolyl and furanyl.

“Heterocycle” or “heterocyclic” or “heterocycloalkyl” or “heterocyclyl” refers include aromatic (also referred to as heteroaryl) and non-aromatic ring compounds containing 3 or more ring members, of which one or more is a heteroatom such as, but not limited to, N, O, and S. In some embodiments, heterocyclyl groups include 3 to 20 ring members, whereas other such groups have 3 to 6, 3 to 10, 3 to 12, or 3 to 15 ring members. Heterocyclyl groups encompass unsaturated, partially saturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. However, the phrase does not include heterocyclyl groups that have other groups, such as alkyl, oxo or halo groups, bonded to one of the ring members. Rather, these are referred to as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic ring compounds containing 5 or more ring members, of which, one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridyl, pyridazinyl, pyrimidinyl, pyrazinyl, thiophenyl, benzothiophenyl, furanyl, benzofuranyl, indolyl, azaindolyl (pyrrolopyridyl), indazolyl, benzimidazolyl, imidazopyridyl (azabenzimidazolyl), pyrazolopyridyl, triazolopyridyl, benzotriazolyl, benzoxazolyl, benzothiazolyl, benzothiadiazolyl, imidazopyridyl, isoxazolopyridyl, thianaphthalenyl, purinyl, xanthinyl, adeninyl, guaninyl, quinolinyl, isoquinolinyl, tetrahydroquinolinyl, quinoxalinyl, and quinazolinyl groups. Although the phrase “heteroaryl groups” includes fused ring compounds such as indolyl and 2,3-dihydro indolyl, the phrase does not include heteroaryl groups that have other groups bonded to one of the ring members, such as alkyl groups. Rather, heteroaryl groups with such substitution are referred to as “substituted heteroaryl groups.” Representative substituted heteroaryl groups may be substituted one or more times with various substituents such as those listed above.

“Stereoisomer” or “stereoisomers” refer to compounds that differ in the chirality of one or more stereocenters. Stereoisomers include enantiomers and diastereomers.

“Enantiomer” refers to one of the two mirror-image forms of an optically active compound.

“Racemate” refers to compounds that contain equal amounts of enantiomers and therefore not being optically active.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2nd ed.; Wiley: New York, 1991).

“Pharmaceutically acceptable salts” refers to relatively non-toxic, inorganic and organic acid addition salts of compositions, including without limitation, analgesic agents, therapeutic agents, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; and the like. See, for example, J. Pharm. Sci., 66:1-19 (1977).

The phrase “pharmaceutically acceptable carrier” is art-recognized, and includes, but is not limited, pharmaceutically acceptable materials, compositions or vehicles, such as a liquid or solid filler, diluent, solvent or encapsulating material, involved in carrying or transporting any subject composition, from one organ, or portion of the body, to another organ, or portion of the body. Each carrier should be “acceptable” in the sense of being compatible with the other ingredients of a subject composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The phrase “therapeutically effective amount” refers to a therapeutically effective, seprase inhibitive amount of a complex or compound of Formula I or II. A therapeutically effective amount can be readily determined by the attending diagnostician, as one skilled in the art, by the use of known techniques and by observing results obtained under analogous circumstances. In determining the therapeutically effective amount or dose, a number of factors are considered by the attending diagnostician, including, but not limited to: the species of mammal; its size, age, and general health; the specific disease involved; the degree of or involvement or the severity of the disease; the response of the individual subject; the particular compound administered; the mode of administration; the bioavailability characteristics of the preparation administered; the dose regimen selected; the use of concomitant medication; and other relevant circumstances.

“Subject” refers to mammals and includes humans and non-human mammals.

“Treating” or “treatment” of a disease in a patient refers to (1) preventing the disease from occurring in a patient that is predisposed or does not yet display symptoms of the disease; (2) inhibiting the disease or arresting its development; or (3) ameliorating or causing regression of the disease.

Radiolabeled derivatives of seprase inhibitors may be used in diagnostic imaging and treatment of diseases that are characterized by the expression of seprase. Identification of compounds that afford affinity and/or selectivity for seprase are also provided. In some aspects, compounds that contain an functionalized proline moiety capable of inhibiting seprase and DPP-IV are incorporated with a chelate-metallic moiety including a radionuclide. In another aspect, compounds that contain an functionalized proline moiety capable of selectively inhibiting seprase over DPP-IV are incorporated with a chelate-metallic moiety including a radionuclide. The radionuclide incorporated into the complex is adapted for radioimaging and/or radiotherapy.

In one aspect, a complex of Formula I, its enantiomer, stereoisomer, racemate or pharmaceutically acceptable salt is provided:

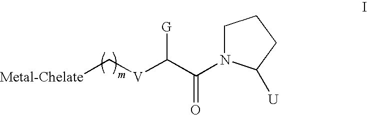

where:
U is selected from the group consisting of —B(OH)₂, —CN, —CO₂H and —P(O)(OPh)₂;
G is selected from the group consisting of H, alkyl, substituted alkyl, carboxyalkyl, heteroalkyl, aryl, heteroaryl, heterocycle and aryl alkyl;
V is a bond, O, S, NH, (CH₂—CH₂—X)ₙ or a group of formula

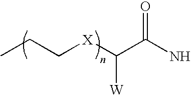

X is O, S, CH₂, or NR;
R is H, Me or CH₂CO₂H; W is H or NHR′;
R′ is hydrogen, acetyl, t-butyloxycarbonyl (Boc), 9H-fluoren-9-ylmethoxycarbonyl (Fmoc), trifluoroacetyl, benzoyl, benzyloxycarbonyl (Cbz) or substituted benzoyl;
n is an integer ranging from 0 to 6;
m is an integer ranging from 0 to 6;
Metal represents a metallic moiety including a radionuclide; and
Chelate represents a chelating moiety that coordinates with said radionuclide.

In some embodiments, Metal includes, but is not limited to, a moiety including a radionuclide. In some embodiments, the moiety is a metal carbonyl. Exemplary radionuclides include, but are not limited to, those of technetium (Tc), rhenium (Re), yttrium (Y), indium (In), and copper (Cu). In some embodiments, the radionuclide is a low oxidation state metal. Examples of low oxidization state metals include metals with an oxidation state less than or equal to about 4, for example Tc(I), Re(I), and Cu(0). In some embodiments, Metal represents a ¹⁸⁵Re-carbonyl, ¹⁸⁶Re-carbonyl, ¹⁸⁸Re-carbonyl, ¹⁸⁵Re-tricarbonyl, ¹⁸⁶Re-tricarbonyl, or ¹⁸⁸Re-tricarbonyl ligand. In some embodiments, Metal represents a ^{99m}Tc-carbonyl ligand or a ^{99m}Tc-tricarbonyl ligand.

Any suitable chelating moiety may be used to provide a covalent or other association with a radionuclide. Examples of chelating agents include but not limited to a substituted or unsubstituted N₂S₂ structure, a N₄ structure, an isonitrile, a hydrazine, a triaminothiol, a chelating agent with a hydrazinonicotinic acid group, a phosphorus group, phosphinothiols, thioesters, thioethers, a picolineamine monoacetic acid, a pyridine or bipyridyl based compound, and a substituted or unsubstituted cyclopentadienyl. By way of example, suitable chelating agents include tetra-azacyclododecanetetra-acetic acid (DOTA), diethylenetriaminepentaacetic acid (DTPA), bis(pyri din-2-ylmethyl)amine (DPA), quinolinemethylamino acetic acid, 2,2′-azanediyldiacetic acid, 2,2′-azanediylbis(methylene)diphenol, 2-((1H-imidazol-2-yl)methylamino)acetic acid, bis(isoquinolinemethyl)amine, bis(quinolinemethyl)amine, pyridine-2-ylmethylamino acetic acid (PAMA), 2-(isoquinolin-3-ylmethylamino)acetic acid, bis((1H-imidazol-2-yl)methyl)amine, bis(thiazol-2-ylmethyl)amine, 2-(thiazol-2-ylmethylamino)acetic acid, and their derivatives, e.g. bis(5-dimethylamino pyridine-2-ylmethyl)amine, bis((1-methyl-/H-imidazol-2-yl)methyl)amine, 2,2′-(2,2′-azanediylbis(methylene)bis(1H-imidazole-2,1-diyl))diacetic acid, 2-((1-(carboxymethyl)-/H-imidazol-2-yl)methylamino)acetic acid, 2,2′-(2-(2-(azanediylbis(methylene)bis(1H-imidazol-1-yl)acetylazanediyl)diacetic acid, and the like. Other chelating groups that may be incorporated into the compound of Formula I, include, but are not limited, to those groups as illustrated in Tables 3 and 4, below.

The distance between the Metal-Chelate moiety and the pyrrolidine moiety of the complex represented by Formula I can be varied by altering the tether and/or expanding the length of the tether between them to modify the affinity and selectivity of the complex for seprase. The pharmacokinetic properties of the complex may also be modified by incorporating heteroatoms into the tethers. The following structures represented by Formulas I-a to I-k are some exemplary embodiments with different tethers and/or the length of tethers. To facilitate description, the complexes are described below with embodiments where the Metal-Chelate moiety has the following structure:

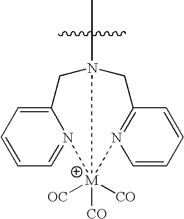

where M is technetium-99m (^{99m}Tc), rhenium-186 (¹⁸⁶Re) or rhenium-188 (¹⁸⁸Re). It will be appreciated that other Metal-Chelate structures are within the scope described embodiments.

In some embodiments, the complex has the structure of Formula I-a:

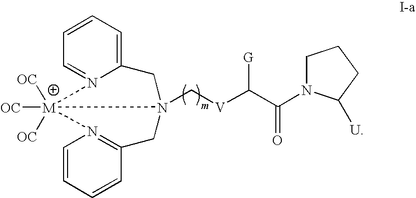

In such embodiments, the variables U, G, V, X, R, W, R′, n, and m are as described above. However, the M is now part of the Metal in Formula I, and M may be a technetium-99m (^{99m}Tc), rhenium-186 (¹⁸⁶Re) or rhenium-188 (¹⁸⁸Re).

In some embodiments, the complex has the structure of Formula I-b:

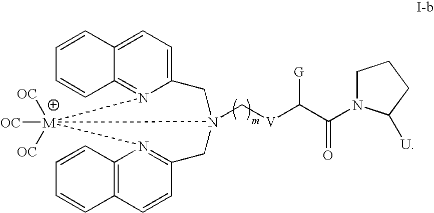

In such embodiments, the variables U, G, V, X, R, W, R′, n, and m are as described above. However, the M is now part of the Metal in Formula I, and M may be a technetium-99m (^{99m}Tc), rhenium-186 (¹⁸⁶Re) or rhenium-188 (¹⁸⁸Re).

In some embodiments, the complex has the structure of Formula I-c:

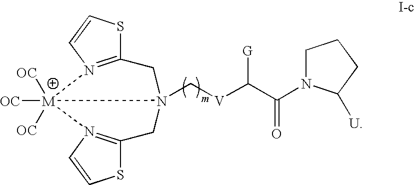

In such embodiments, the variables U, G, V, X, R, W, R′, n, and m are as described above. However, the M is now part of the Metal in Formula I, and M may be a technetium-99m (^{99m}Tc), rhenium-186 (¹⁸⁶Re) or rhenium-188 (¹⁸⁸Re).

In some embodiments, the complex has the structure of Formula I-d:

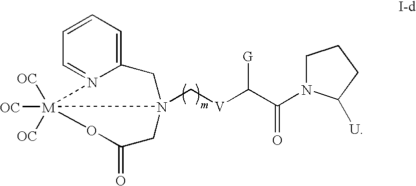

In such embodiments, the variables U, G, V, X, R, W, R′, n, and m are as described above. However, the M is now part of the Metal in Formula I, and M may be a technetium-99m (^{99m}Tc), rhenium-186 (¹⁸⁶Re) or rhenium-188 (¹⁸⁸Re).

In some embodiments, the complex has the structure of Formula I-e:

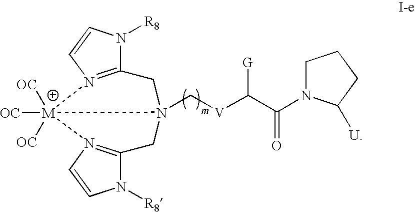

In such embodiments, the variables U, G, V, X, R, W, R′, n, and m are as described above. However, the M is now part of the Metal in Formula I, and M may be a technetium-99m (^{99m}Tc), rhenium-186 (¹⁸⁶Re) or rhenium-188 (¹⁸⁸Re). Furthermore, R₈ and R₈′ are independently hydrogen, halogen, a substituted or unsubstituted alkyl, alkenyl, alkynyl, hydroxyl, alkoxyl, acyl, acyloxy, acylamino, silyloxy, amino, monoalkylamino, dialkylamino, nitro, sulfhydryl, alkylthio, imino, amido, phosphoryl, phosphonate, phosphine, carbonyl, carboxyl, carboxamide, anhydride, silyl, thioalkyl, alkylsulfonyl, aryl sulfonyl, selenoalkyl, ketone, aldehyde, ether, ester, heteroalkyl, cyano, guanidine, amidine, acetal, ketal, amine oxide, aryl, heteroaryl, aralkyl, arylether, heteroaralkyl, azido, aziridine, carbamoyl, epoxide, hydroxamic acid, imide, oxime, sulfonamide, thioamide, thiocarbamate, urea, thiourea, (CH₂)_{d}CO₂H, CH₂CH₂OCH₂CH₃, CH₂CH(OCH₃)₂, (CH₂CH₂O)_{d}CH₂CH₃, (CH₂)_{d}NH₂, CH₂CH₂C(O)NH₂, (CH₂)_{d}C(O)N((CH₂)_{d}COOH)₂, (CH₂)_{d}N(CH₃)₂, CH₂CH₂OH, (CH₂)_{d}CH(CO₂H)₂, (CH₂)_{d}P(O)(OH)₂, (CH₂)_{d}B(OH)₂, or —(CH₂)_{d}—R₉, where each d is individually an integer from 0 to 6; and each R₉ is independently 15-Crown-5,18-Crown-6, tetrazole, oxazole, aziridine, triazole, imidazole, pyrazole, thiazole, hydroxamic acid, phosphonate, phosphinate, thiol, thioether, polysaccharide, sacharride, nucleotide or oligonucleotide. In some embodiments, R₈ and R₈′ are (CH₂)_{d}C(O)N((CH₂)_{d}COOH)₂. In some embodiments, R₈ and R₈′ are CH₂C(O)N(CH₂COOH)₂. In some embodiments, R₈ and R₈′ are (CH₂)_{d}COOH. In some embodiments, R₈ and R₈′ are CH₂COOH.

In some embodiments, the complex has the structure of Formula I-f:

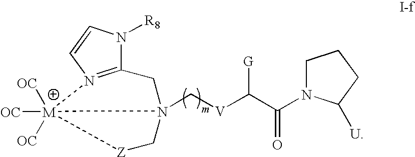

In such embodiments, the variables U, G, V, X, R, W, R′, R₈, n, and m are as described above. However, the M is now part of the Metal in Formula I, and M may be a technetium-99m (^{99m}Tc), rhenium-186 (¹⁸⁶Re) or rhenium-188 (¹⁸⁸Re). In such embodiments, Z is a substituted or unsubstituted thioalkyl, carboxylate, carboxyalkyl, aminoalkyl, heterocyclyl, (amino acid), (amino acid)alkyl, hydroxy, hydroxyalkyl, 2-(carboxy)aryl, 2-(carboxy)heteroaryl, 2-(hydroxy)aryl, 2-(hydroxy)heteroaryl, 2-(thiol)aryl, 2-pyrrolidine boronic acid, or 2-(thiol)heteroaryl.

In some embodiments, the complex has the structure of Formula I-g:

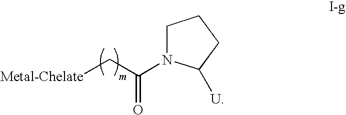

In such embodiments, U, m, Metal, and Chelate are as described above. In some embodiments, the Metal is metal containing moiety, that contains technetium-99m, rhenium-186 or rhenium-188.

In some embodiments, the complex has the formula of I-h:

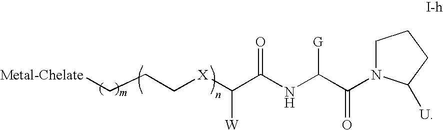

In such embodiments, the variables U, G, X, R, W, R′, n, m, and Chelate are as described above. However, the metal in the Metal moiety in Formula I may be technetium-99m (^{99m}Tc), rhenium-186 (¹⁸⁶Re) or rhenium-188 (¹⁸⁸Re).

In some embodiments, the complex has the formula of I-i:

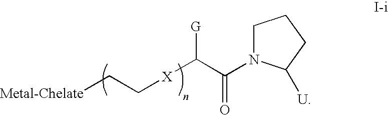

In such embodiments, the variables U, G, X, n and Chelate are as described above. However, the metal in the Metal moiety in Formula I may be technetium-99m (^{99m}Tc), rhenium-186 (¹⁸⁶Re) or rhenium-188 (¹⁸⁸Re).

In another aspect, iodinated analogs of compounds that show selectivity of seprase over DPP-IV are provided. Structure activity relationship can be developed on the selective compounds to provide iodinated analogs for radioiodination. Provided below is a compound of general Formula II, its enantiomer, stereoisomer, racemate or pharmaceutically acceptable salt:

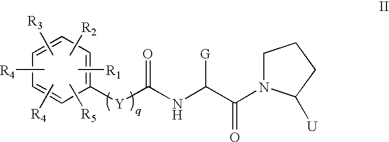

where:
U is —B(OH)₂, —CN, —CO₂H, or —P(O)(OPh)₂;
G is H, alkyl, substituted alkyl, carboxyalkyl, heteroalkyl, aryl, heteroaryl, heterocycle, or arylalkyl;
Y is a bond, —O—, —CH₂—, —OCH₂—, —CH₂O—, NR, —NR—CH₂, or CH₂—NR—, wherein R is H, Me or CH₂CO₂H;
q is an integer ranging from 0 to 24; and
R₁, R₂, R₃, R₄ and R₅ are independently hydrogen, halogen, cyano, carboxyl, alkyl, alkylamino, alkoxy, or substituted or unsubstituted amino, provided that at least one of R₁, R₂, R₃, R₄ and R₅ is a radiohalogen.

In some embodiments, the radiohalogen is selected radioiodine or radiofluorine.

In some embodiments, the compound has the structure of Formula II-a:

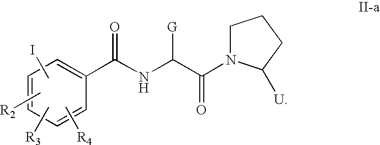

In such embodiments, U and G are as described above. In such embodiments, R₂, R₃, and R₄ are independently H, halogen, cyano, carboxyl, alkyl, alkylamino, alkoxy, or substituted or unsubstituted amino; and I is a radioiodine.

In some embodiments, the compound has the structure of Formula II-b:

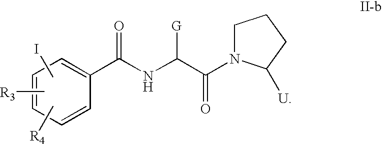

In such embodiments, U and G are as described above. In such embodiments, R₃, and R₄ are independently H, halogen, cyano, carboxyl, alkyl, alkylamino, alkoxy, or substituted or unsubstituted amino; and I is a radioiodine.

In some embodiments, the compound has the structure of Formula II-c:

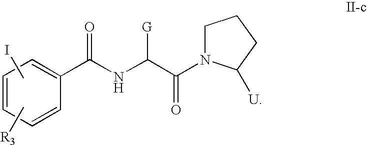

In such embodiments, U and G are as described above. In such embodiments, R₄ is H, halogen, cyano, carboxyl, alkyl, alkylamino, alkoxy, or substituted or unsubstituted amino; and I is a radioiodine.

In some embodiments, the compound has the structure of Formula II-d:

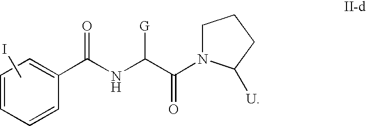

In such embodiments, U and G are as described above, and I is a radioiodine.

The complex or compound represented by Formula I and II, its enantiomer, stereoisomer, racemate or pharmaceutically acceptable salt, may be prepared by methods known in the art. In general, the complex represented by Formula I may be prepared by incorporating a Metal-Chelate moiety into a compound containing boronoproline, pyrrolidine-2-carbonitrile, proline or phosphoproline moiety that exhibits selective binding to seprase over DPP-IV.

By way of example, the Metal-Chelate compounds may be made by Single Amino Acid Chelate (SAAC™) technology, as described in U.S. Patent Application Publication No. 2003/0235843. A variety of structurally diverse molecules can be made using the SAAC technology. The SAAC technology may provide a rapid, high yield, one pot synthesis of mono-, di-, and mixed alkylated amino acid derivatives. The alkylated amino acid derivatives may possess a tridentate chelating moiety distal to an amino acid functionality. The tridentate chelating group allows facile and robust coordination of a metallic moiety or metallic core such as {M(CO)₃}⁺¹ core (M is a radionuclide such as Tc or Re). In some embodiments, a metallic core may be inserted prior to performing standard chemistries, including standard deprotection and peptide cleavage chemistries, without loss of the metal from the SAAC complex. Studies on the coordination chemistry of the {M(CO)₃}⁺¹ core have established that amine, aromatic, heterocyclic, and carboxylate donors provide effective chelating ligands. The tridentate chelate-M(CO)₃ complexes provide chemical inertness and a broad utility of the amino acid functionality. Various tridentate chelating moieties can be made so as to alter the charge, hydrophobicity, and distance of the tridentate chelate-M(CO)₃ complex from the functional moiety of the compound. Scheme 1 illustrates the preparation of examples of alkylated SAAC molecules by direct reductive N-alkylations of t-butyloxycarbonyl (BOC) protected lysine with the desired aldehydes with NaBH(OAc)₃ as the reducing agent.

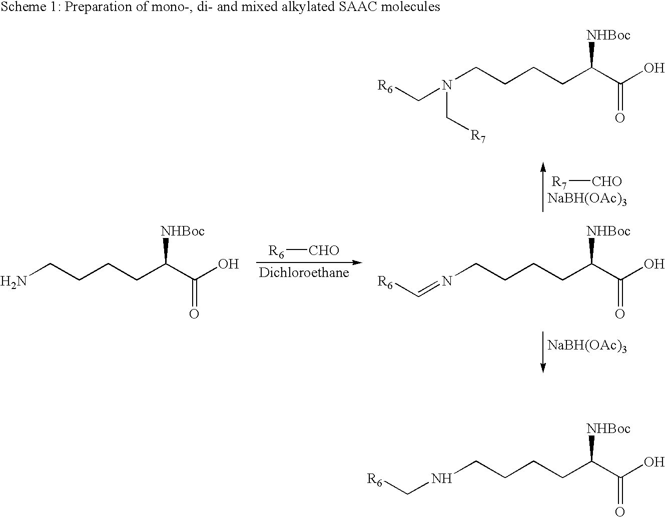

wherein R₆ and R₇ are independently selected from the group consisting of a-g.

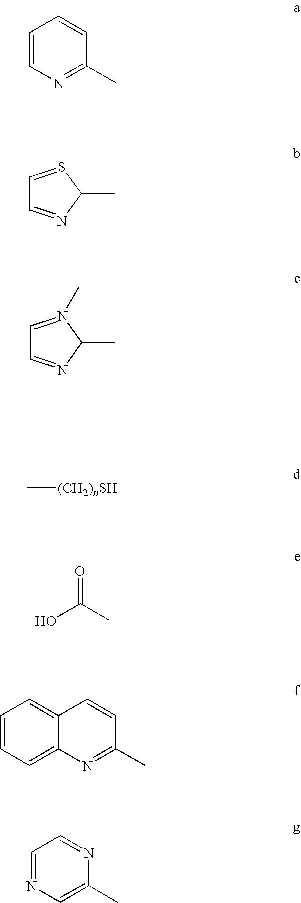

The {M(CO)₃}⁺¹ (M is e.g. Tc or Re) complexes of the bifunctional chelates can be readily prepared from, for example, and [Et₄N]₂[Re(CO)₃Br₃], [Re(CO)₃(H₂O)₃]Br, or [Tc(CO)₃(H₂O)₃]. Such metal carbonyl compounds may be generated in situ from the commercially available tricarbonyl kits (Mallinckrodt).

Scheme 2 illustrates the synthesis of boronoproline-M⁺(CO)₃ complexes having the structure of Formula I. The effect of the different chelating groups of the metal complex on the inhibition and selectivity of seprase over DPP-IV can be investigated with the exemplary complexes.

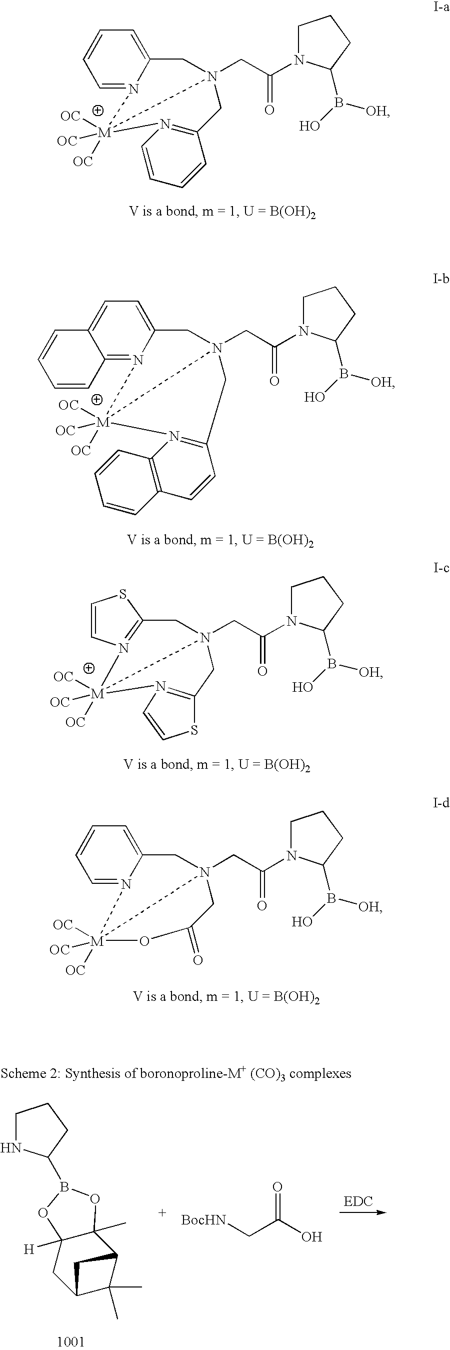

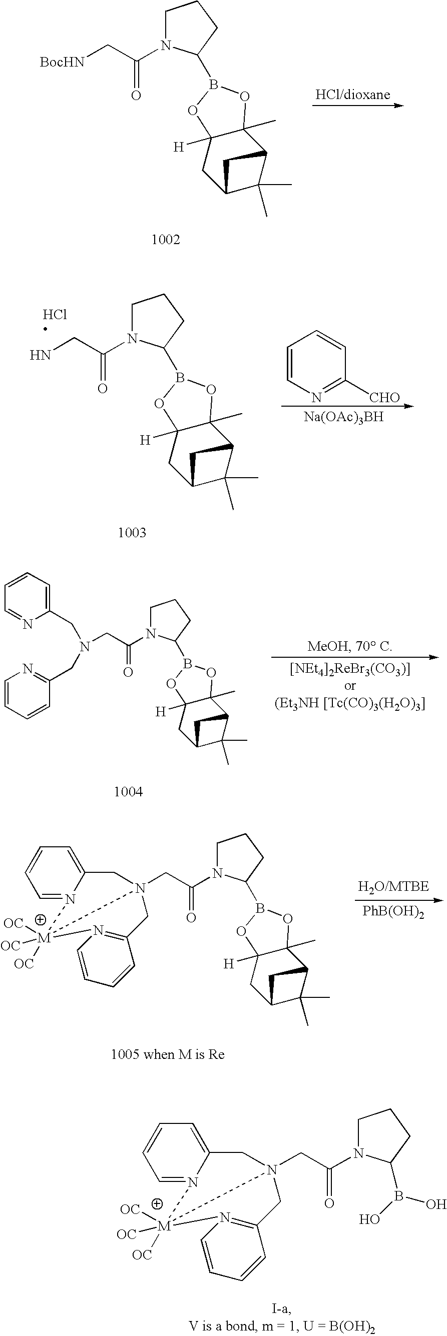

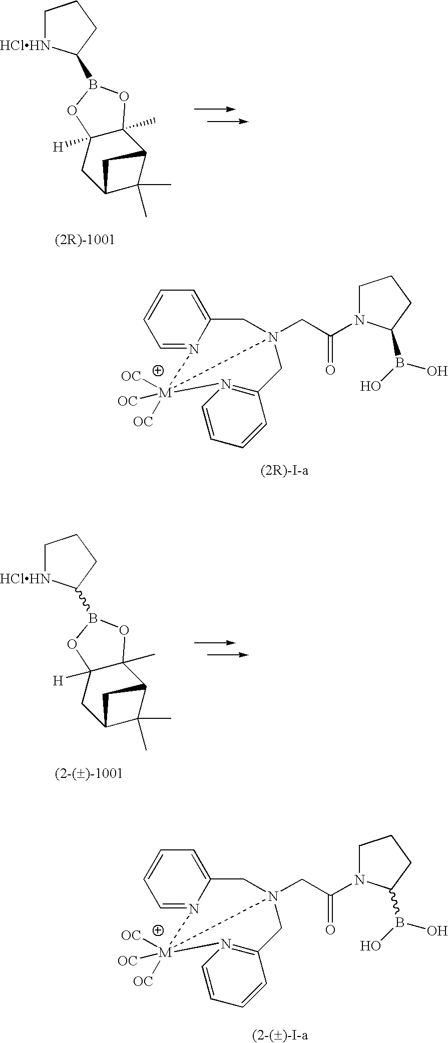

The syntheses can be accomplished by reductive amination of the borane-protected boronoproline 1003 with two equivalents of the appropriate aldehyde (e.g. 2-pyridinecarboxaldehyde) using sodium triacetoxyborohydride as the reducing agent. The free ligands thus obtained may then be complexed with the desired metal followed by removal of borane protection group to afford the desired metal complex I-a. Similarly, compounds of Formula I-a with U=P(O)(OPh)₂, CO₂H or CN can be prepared when diphenyl pyrrolidin-2-ylphosphonate, proline or pyrrolidine-2-carbonitrile is used as a starting material, respectively. The borane-protected boronoproline 1003 can be prepared from the corresponding compound 1001 via standard peptide formation. One skilled in the art would readily utilize any appropriate chiral or non-chiral borane-protecting groups to prepare compound 1001 in racemic or enantiomeric form according to the known procedures (see examples in Coutts et al., J. Med. Chem. 1996, 39(10), 2087-2094). Compounds of Formula I-a in racemic or enantiomeric form can then be prepared accordingly.

Scheme 3 illustrates the synthesis of functionalized proline-M⁺(CO)₃ complex (e.g. U=B(OH)₂) from a pre-formed M⁺(CO)₃ ligand. The synthetic route utilizes the pre-formed chelate and enantiomerically rich boronoproline 1001 as the starting materials to prepare the boronoproline M(CO)₃ Dpa analog (1-g, wherein m=5, Chelate is Dpa, Metal=Re or Tc). Compounds of Formula I-g in racemic form can be prepared utilizing a non-chiral starting material, e.g. a racemate analog of compound 1001.

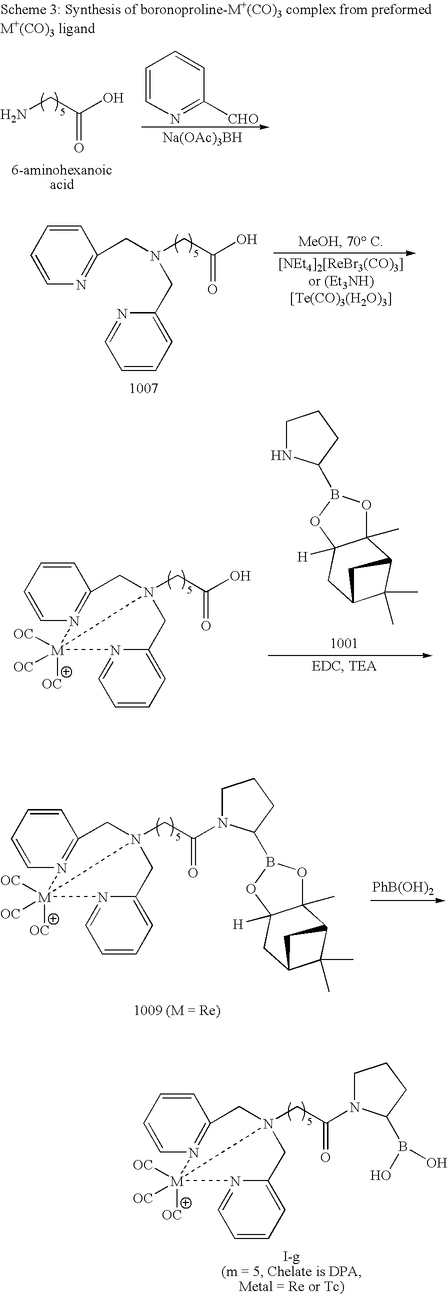

Similarly, compounds of Formula I-g with U=P(O)(OPh)₂, CO₂H or CN can be prepared where diphenyl pyrrolidin-2-ylphosphonate, proline or pyrrolidine-2-carbonitrile is used as a starting material, respectively.

Scheme 3 can be utilized to synthesize functionalized proline-M⁺(CO)₃ complexes to explore the effect of more significant variations of the distance of the metal chelator from the proline moiety by incorporating a tether into these structures. The tether may comprise a simple alkyl chain as shown, a PEG (CH₂CH₂O)ₙ, a polyethylene amine ((CH₂CH₂NH)ₙ), or the like. Terminal aminoalkanoic acids (such as (β-alanine, 4-aminobutanoic acid, 5-aminopentanoic acid, 6-aminohexanoic acid and the 8-aminooctanoic acid) or amino-PEG-acids (NH₂—(CH₂CH₂O)ₙ—CH₂—COOH, e.g. 2-(2-(3-aminopropoxy)ethoxy)acetic acid) may be utilized as tethers, according to some embodiments.

According to some embodiments, glycine and/or other appropriate amino acid can be incorporated as a linker, as well as an additional binding moiety to afford seprase inhibitors. Scheme 4 illustrates synthesis of boronoproline-Re⁺(CO)₃ or Tc⁺(CO)₃ complexes having the structure as represented by Formula I-b and I-c. Lysine is used to prepare compounds of Formula I with additional amine moiety in the linkers.

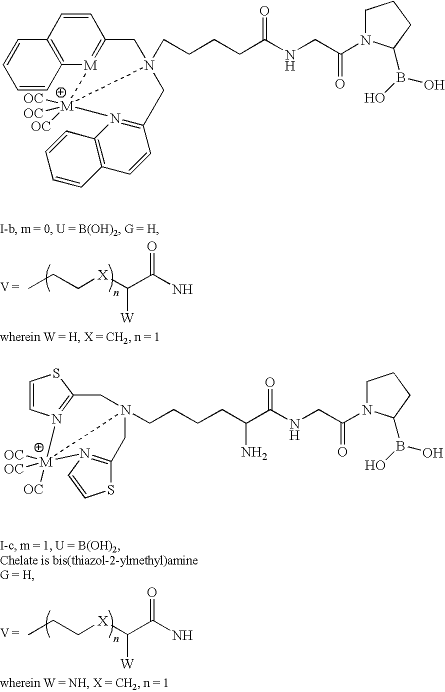

The molecules in this class may be prepared from the corresponding compound 1003, as illustrated in Scheme 4. The complexes of Formula I-b can be prepared from protected lysine utilized standard peptide coupling chemistry. The complexes of Formula I-c are prepared from the appropriate terminal aminoalkanoic acids. Similarly, by employing suitable borane-protecting groups, one can prepare the corresponding racemic compound (from the non-chiral borane-protecting starting material) or the enantiomer (from the chiral-protecting starting material in opposite chirality).

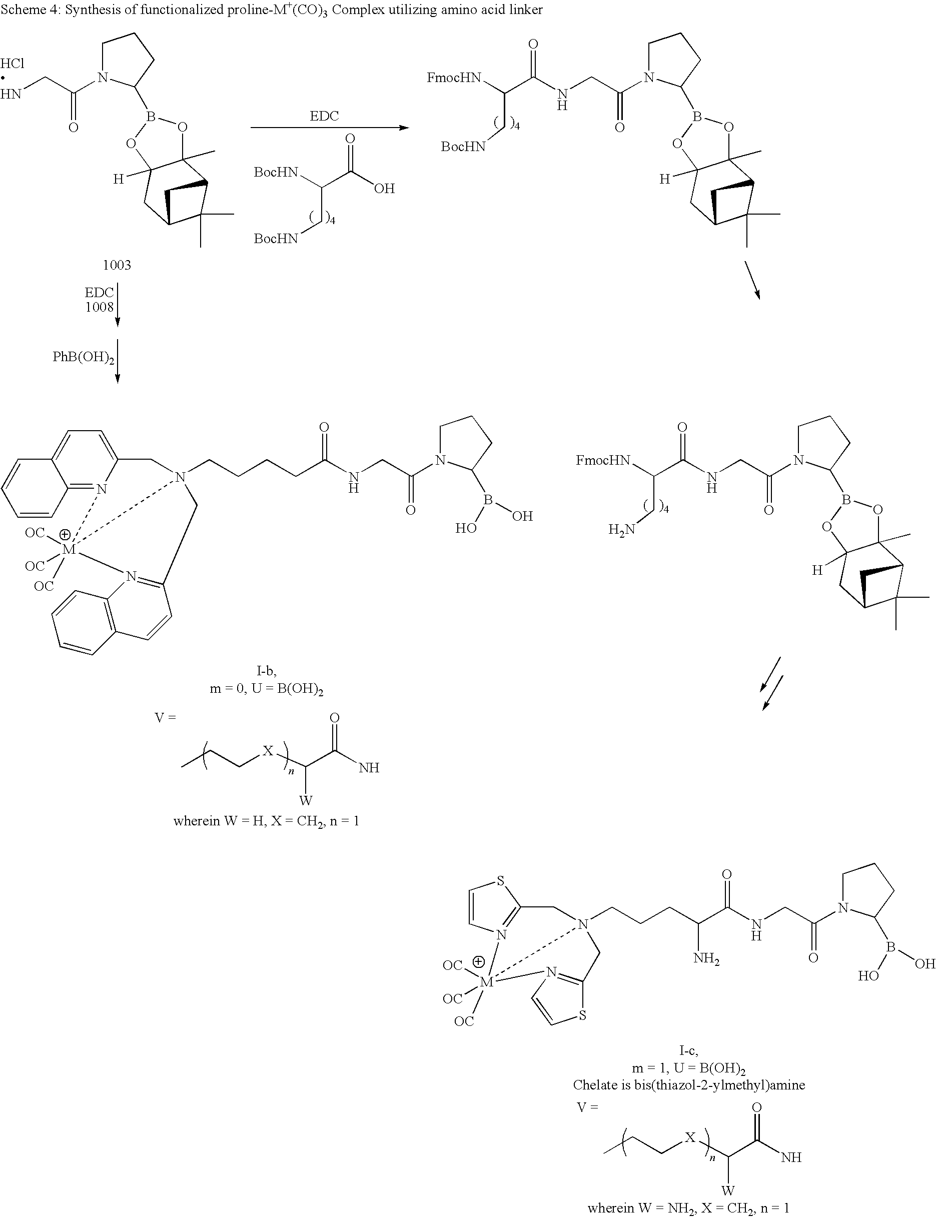

The above reaction scheme is applicable to any modification of the tether by incorporation of heteroatoms into the tether chain. This may have additional benefits on the affinity as well as the selectivity for seprase. Incorporation of heteroatoms into the tether such as oxygen can take advantage of the commercially availability of a variety of short polyethylene glycol (PEG) diamines that can be readily incorporated into the complexes. One of ordinary skilled in the art would also readily apply other chelates to prepare functionalized proline-M⁺(CO)₃ complexes.

The general synthesis of the N-substituted benzamide glycineboronoproline analogs is shown below in Scheme 5. The synthesis of the glycineboronoproline intermediate has been previously described (Simon J. C. et al. J. Med. Chem. 1996, 39, 2087-2094). The two step synthesis from compound 1003 (or its racemic or enantiomeric analogs) involves an amide formation and deprotection steps. Similarly, compounds of Formula II with U=P(O)(OPh)₂, CO₂H or CN can be prepared when diphenyl pyrrolidin-2-ylphosphonate, proline or pyrrolidine-2-carbonitrile is used as a starting material, respectively to afford the corresponding compound 1003 analogs via the glycine coupling steps as described before. Alternatively, compound 1001 may be used to couple with glycine-linked N-substituted benzamide for the preparation of compound of general Formula IV.

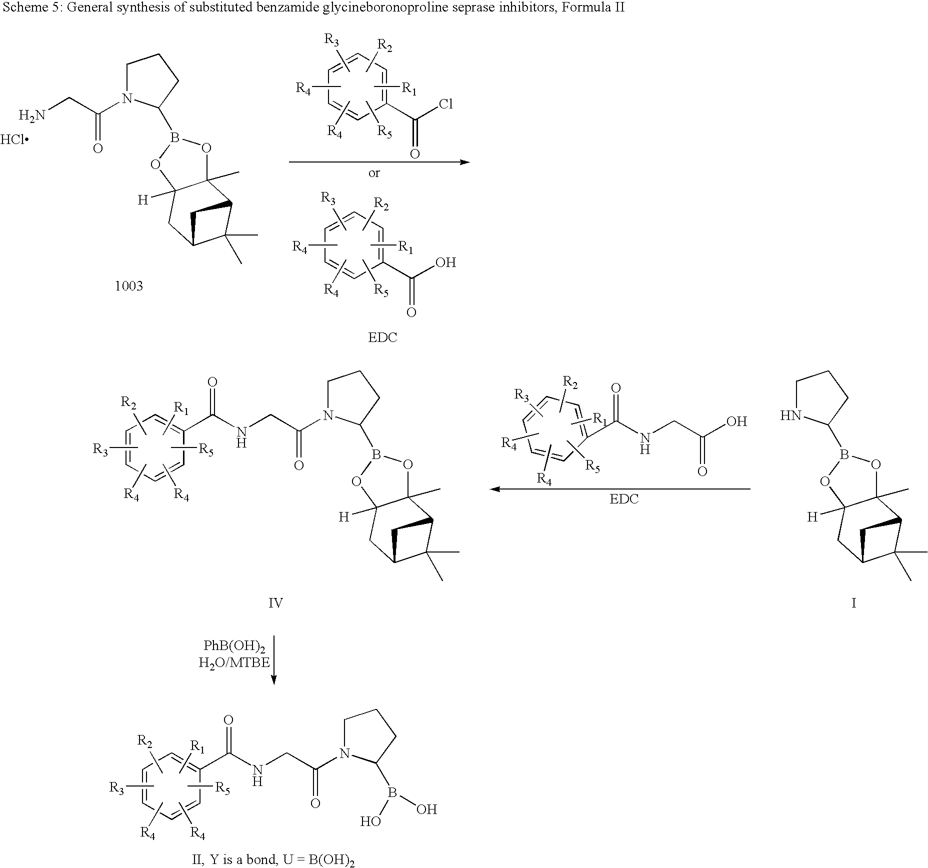

The synthesis of the radioiodinated analogs may be prepared by a direct iododestannylation of the stannylated boronic acid or boronate (i.e. compound IV-a) or as shown by the direct iododestannylation of the boronic acid in Scheme 6, although the undesirable iododeboronation reaction may lower the yield. It may also proceed via a two step process wherein the iododestannylation is first conducted on a stannylated benzoic acid which is then coupled to the glycineboronoproline intermediate as either the boronic acid or the boronate (Scheme 6).

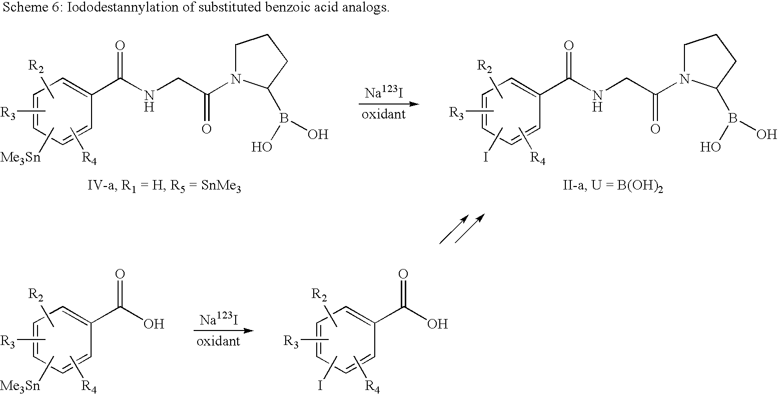

The complexes or compounds, may be used in accordance with the methods also described herein, by those skilled in the art, e.g., by specialists in nuclear medicine, for diagnostic imaging of tissue which expresses seprase, and therapeutic treatment of diseases which are characterized by overexpression of seprase.

The complexes or compounds may be used in the following manner. An effective amount of the compound (from 1 to 50 mCi) may be combined with a pharmaceutically acceptable carrier for use in imaging studies. As used herein, “an effective amount” of the compound is defined as an amount sufficient to yield an acceptable image using equipment which is available for clinical use. An effective amount of the complex may be administered in more than one injection. Effective amounts of the complex will vary according to factors such as the degree of susceptibility of the individual, the age, sex, and weight of the individual, idiosyncratic responses of the individual, and dosimetry. Effective amounts of the complex will also vary according to instrument and film-related factors. Optimization of such factors is well within the level of skill of a person skilled in the art.

As used herein, the pharmaceutically acceptable carrier includes any and all solvents, dispersion media, coatings, antibacterial and antifungal agents, isotonic agents, absorption delaying agents, and the like. The use of such media and agents for pharmaceutically active substances is well known in the art. The complex or compound may be administered to an individual in an appropriate diluent or adjuvant, or in an appropriate carrier such as human serum albumin or liposomes. Supplementary active compounds can also be used with the complex. Pharmaceutically acceptable diluents include saline and aqueous buffer solutions. Adjuvants contemplated herein include resorcinols, non-ionic surfactants such as polyoxyethylene oleyl ether and hexadecyl polyethylene ether.

In one embodiment, the complex or compound, its enantiomer, stereoisomer, racemate or pharmaceutically acceptable salt, is administered parenterally as injections (intravenous, intramuscular or subcutaneous). The complex or compound, its enantiomer, stereoisomer, racemate or pharmaceutically acceptable salt, may be formulated as a sterile, pyrogen-free, parenterally acceptable aqueous solution. The preparation of such parenterally acceptable solutions, having due regard to pH, isotonicity, stability, and the like, is within the skill in the art. Certain pharmaceutical compositions suitable for parenteral administration include one or more imaging agents in combination with one or more pharmaceutically acceptable sterile powders which may be reconstituted into sterile injectable solutions or dispersions just prior to use. The pharmaceutical compositions may also contain antioxidants, buffers, bacteriostats, solutes which render the formulation isotonic with the blood of the intended recipient, or suspending or thickening agents. A formulation for injection may contain, in addition to the imaging agent, an isotonic vehicle such as sodium chloride solution, Ringer's solution, dextrose solution, dextrose and sodium chloride solution, lactated Ringer's solution, dextran solution, sorbitol solution, a solution containing polyvinyl alcohol, or an osmotically balanced solution including a surfactant and a viscosity-enhancing agent, or other vehicle as known in the art. The formulations may also contain stabilizers, preservatives, buffers, antioxidants, or other additives known to those skilled in the art.

The amount of the complex or compound, its enantiomer, stereoisomer, racemate or pharmaceutically acceptable salt, used for diagnostic or therapeutic purposes may depend upon the nature and severity of the condition being treated, on the nature of therapeutic treatments which the patient has undergone, and on the idiosyncratic responses of the patient. Ultimately, the attending physician will decide the amount of complex or compound to administer to each individual patient and the duration of the imaging study.

In another aspect, a kit is provided for imaging which includes one or more of the complex(es) or compound(s), its enantiomer, stereoisomer, racemate or pharmaceutically acceptable salt, described above, in combination with a pharmaceutically acceptable solution containing a carrier such as human serum albumin or an auxiliary molecule such as mannitol or glaciate. Human serum albumin for use in the kit may be made in any way, for example, through purification of the protein from human serum or through recombinant expression of a vector containing a gene encoding human serum albumin. Other substances may also be used as carriers, for example, detergents, dilute alcohols, carbohydrates, and the like. In one embodiment, a kit may contain from about 1 to about 50 mCi of a complex or compound, its enantiomer, stereoisomer, racemate or pharmaceutically acceptable salt. In another embodiment, a kit may contain the unlabeled fatty acid stereoisomer which has been covalently or non-covalently combined with a chelating agent, and an auxiliary molecule such as mannitol, gluconate, and the like. The unlabeled fatty acid stereoisomer/chelating agent may be provided in solution or in lyophilized form. The kits may also include other components which facilitate practice of the described methods. For example, buffers, syringes, film, instructions, and the like may optionally be included as components of the kits of the disclosure.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology, thus generally described, will be understood more readily by reference to the following examples, which are provided by way of illustration and are not intended to be limiting in any way.

### EXAMPLES

In the following examples, reactions are carried out in dry glassware under an atmosphere of argon or nitrogen unless otherwise noted. Reactions are purified by flash column chromatography, medium pressure liquid chromatography or by preparative high pressure liquid chromatography (HPLC). ¹H NMR will be obtained on a Bruker 400 MHz instrument. Spectra are reported as ppm δ and are referenced to the solvent resonances in CDCl₃, DMSO-d₆ or methanol-d₄. Solvents and reagents are obtained from commercial sources.

The following abbreviations are used in the examples: dichloromethane (DCM), ethyl acetate (EA), hexanes (Hex), dichloroethane (DCE), dimethyl formamide (DMF), methyl tent-butyl ether (MTBE), trifluoroacetic acid (TFA), tetrahydrofuran (THF), carbonyldiimidazole (CDI), dicyclohexyl carbodiimide (DCC), dimethylaminopyridine (DMAP), t-butyloxycarbonyl (BOC), diisopropylethylamine (DIPEA), triethylamine (TEA), benzyloxycarbonyl (CBZ), phenylboronic acid (PhB(OH)₂), ethanol (EtOH), 1-Ethyl-3-[3-dimethylaminopropyl]carbodiimide Hydrochloride (EDC or EDCI) and methanol (MeOH). If not defined, the abbreviations or terms have their generally accepted meanings.

### Example 1

### Preparation of a glycine-boronoproline Intermediate, Compound 1003

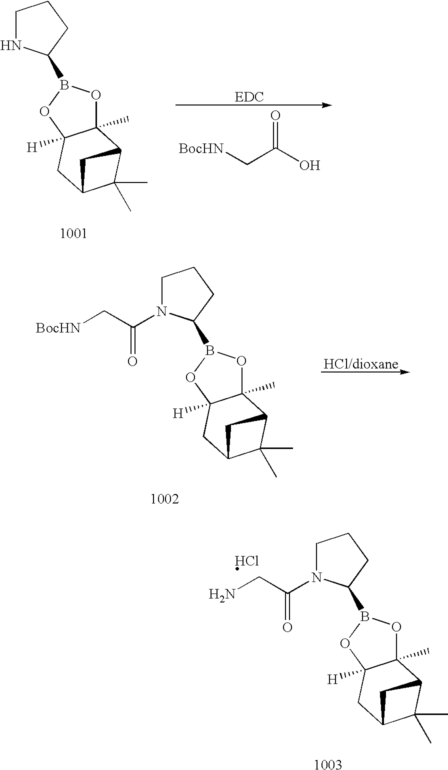

The amino boronic ester 1001 can be prepared according to literature procedure (Coutts S. J. et al. J. Med. Chem. 1996, 39, 2087). Boc-glycine-OH is to be coupled with 1001 in the presence of EDC at room temperature to generate the fully protected dipeptide 1002. The Boc group can be removed with HCl in dioxane to produce the unprotected amine 1003. Similarly, by employing a non-chiral borane protecting group (e.g. a non-chiral diol) or an enantiomer of the chiral borane protecting group, one skilled in the art could prepare compound 1003 analogs in racemic form or in opposite enantiomeric form.

### Example 2

### Reductive Amination of Glycine-Boronoproline Intermediates

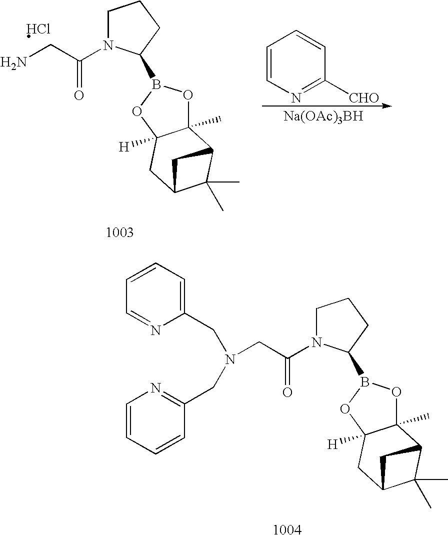

The unprotected amine 1003 (1 equivalent) is added to DCE or other suitable solvent and vigorously stirred at room temperature, and 2-pyridine carboxaldehyde (2 to 3 equivalents) is added in one portion. The solution is then stirred for 10 to 30 min at room temperature, followed by the addition of sodium triacetoxyborohydride (2.2 to 3.2 equivalents) in one portion. The solution is stirred overnight at room temperature. The solution is then evaporated to dryness, treated with 2N sodium hydroxide aqueous solution, and extracted with DCM. The organic extracts are dried over sodium sulfate and concentrated to afford compound 1004.

Other aldehydes (e.g. isoquinoline-1-carbaldehyde, thiazole-2-carbaldehyde, etc.) may be used to similarly prepare intermediates having the desired chelating groups.

### Example 3

### Preparation of Rhenium (I) Complexes

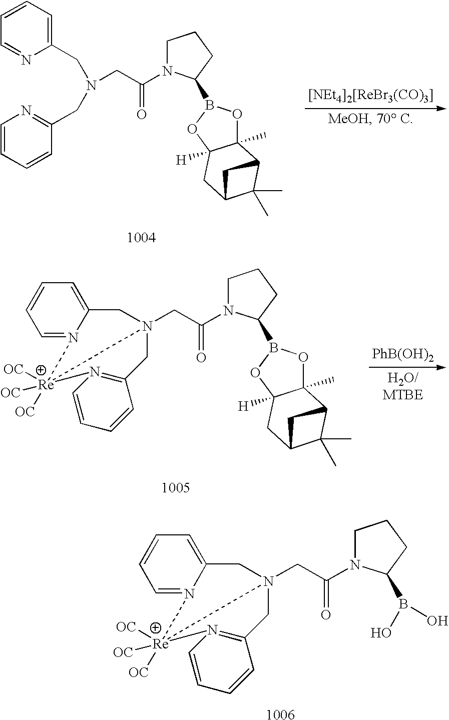

A suspension of 1004 (1 equivalent) and Re(CO)₃(H₂O)₂Br, or (NEt₄)₂ReBr₃(CO)₃, (1.1 equivalent), in MeOH, or other suitable solvent is placed in a pressure tube. The reaction mixture is heated on an oil bath at elevated temperature (e.g. 100-125° C.) for 36 hours, or more, and then cooled to room temperature. The resulting suspension is then diluted with water and extracted with DCM, or other suitable organic solvents. The extracts are the applied to a pad of silica gel, and eluted with MeOH (10%) in DCM as the eluent. The solvents are removed in vacuo, and the residue crystallized from water-methanol to afford boronic ester 1005.

Deprotection of the boronic ester 1005 is achieved by transesterification of the pinanediol with phenylboronic acid in a biphasic MTBE-water mixture. Pinanediol phenylborate is recovered from the organic phase, and the desired compound 1006 is isolated from the aqueous phase under suitable conditions.

### Example 4

### Preparation of Metal-Chelates

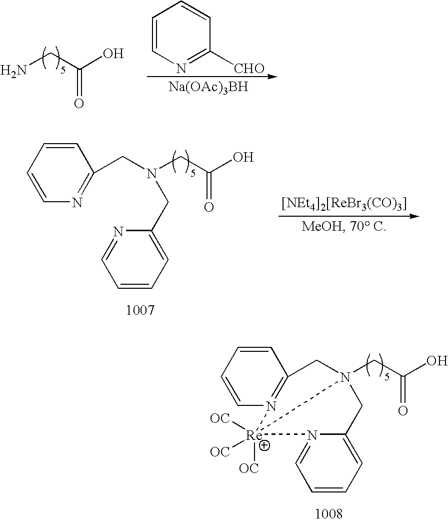

Commercially available 6-aminohexanoic acid (1 equivalent) is added to DCE and vigorously stirred at room temperature while 2-pyridine carboxaldehyde (2.2 equivalent) is added in one portion. The solution is stirred for 10 to 30 min at room temperature, then sodium triacetoxyborohydride (2.5 equivalent) is added in one portion. The solution is stirred overnight at room temperature. Upon completion, the solution is evaporated to dryness, treated with 2N sodium hydroxide aqueous solution, and extracted with DCM. The organic extracts are dried over sodium sulfate and concentrated to afford compound 1007.

Compound 1007 (1.0 equivalent) is dissolved in methanol in a pressure tube and Re(CO)₃(H₂O)₂Br (1.1 equivalent) is added and stirred under argon overnight or longer at elevated temperature (e.g. 100-125° C.). The solution is concentrated under vacuum and treated with acetone, or other suitable solvent(s), and filtered through Celite. The solution is then evaporated to afford the desired product 1008.

### Example 5

### Preparation of a Compound Formula I-g, as Exemplified by Re(CO)₃[1-(6-(bis(pyridin-2-ylmethyl)amino)hexanoyl)pyrrolidin-2-ylboronic acid] (1010)

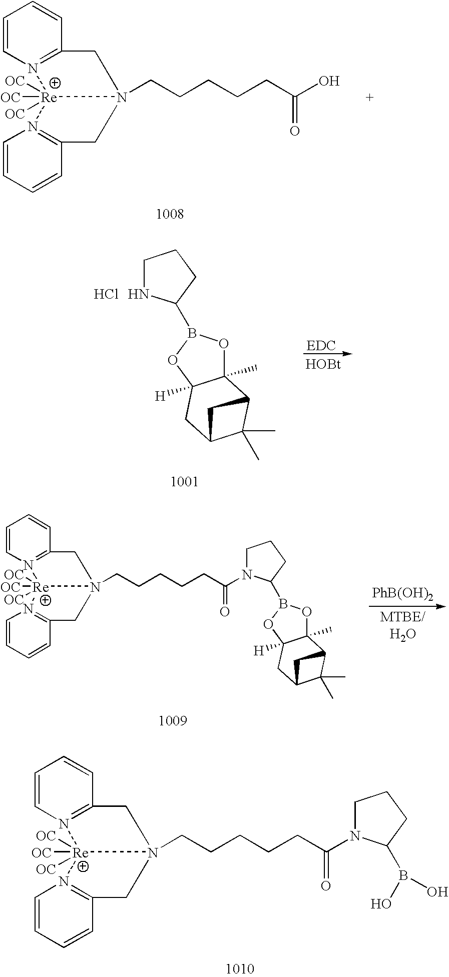

The metal-chelate 1008 was coupled with compound 1001 under the similar conditions described above to afford compound 1009. Deprotection of boronic ester 1009, results in compound 1010. Re(CO)₃[1-(6-(bis(pyridin-2-ylmethyl)amino)hexanoyl)pyrrolidin-2-ylboronic acid] (1010), ESI MS m/z 681 (M+H⁺); ¹H NMR (400 Hz, CD₃OD): δ 8.76 (d, J=17.0 Hz, 2H), 7.84 (t, J=20.0, 2H), 7.43 (d, J=20.0 Hz, 2H), 7.27 (t, J=17.0 Hz, 2H), 3.72 (m, 2H), 3.57 (m, 2H), 3.47 (m, 2H), 3.20 (m, 5H), 2.40 (m, 2H), 1.89 (m, 2H), 1.67 (m, 3H), 1.40 (m, 3H) (boronic acid OH's not seen).

### Example 6

### Preparation of Complexes of Formula I Via Amidation

Complexes of Formula I may be prepared by amidation of a chelate or metal-chelate. For example, compound 1003 is coupled with metal-chelate 1008, or the like, in the presence of EDC and DIPEA, at room temperature, to generate a protected intermediate (e.g. a boronic ester), followed by deprotection of the boronic ester to afford final product 1011.

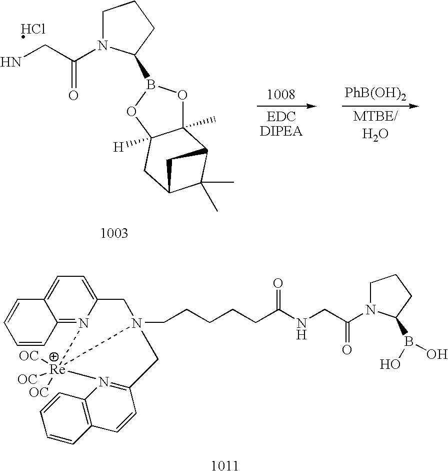

The chelate-rhenium boronic esters were prepared following the Scheme 1 shown below. The end products were isolated from aqueous phase by reverse phase HPLC, after the deprotection of the boronic esters. Various chelates were used to provide chelate-rhenium boronic esters.

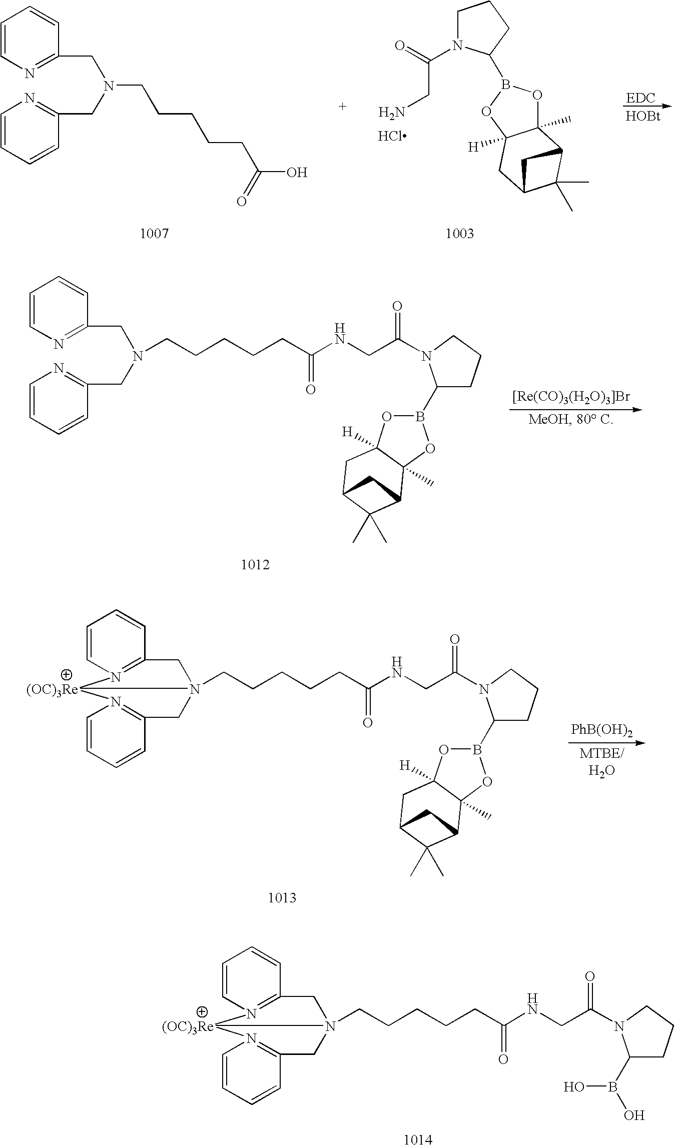

The compounds of the above synthetic scheme and their characterization data include, 1-(2-(6-(bis(pyridin-2-ylmethyl)amino)hexanamido) cetyl)pyrrolidin-2-ylpinadiol borate (1012), ESI MS m/z 602 (M+H⁺); and Re(CO)₃[1-(2-(6-(bis(pyridin-2-ylmethyl)amino)hexanamido) acetyl)pyrrolidin-2-ylboronic acid] (1014), ESI MS m/z 739 (M+H⁺); ¹H NMR (400 Hz, CD₃OD): δ 8.86 (d, J=13.0 Hz, 2H), 7.94 (m, 2H), 7.54 (m, 2H), 7.37 (d, J=13.0 Hz, 2H), 4.84 (s, 4H), 3.93 (m, 2H), 3.46-3.63 (m, 3H), 3.35 (m, 5H), 2.32 (m, 2H), 2.16 (bs, 1H), 1.94 (m, 4H), 1.74 (m, 2H), 1.50 (m, 2H), (amide NH's not seen).

Other exemplary compounds that may be prepared via similar methods include:

2-((6-(2-(2-(pinadioxyboryl)pyrrolidin-1-yl)-2-oxoethylamino)-6-oxohexyl)(pyridin-2-ylmethyl)amino)acetic acid (1015), ESI MS m/z 569.0 (M+H⁺):

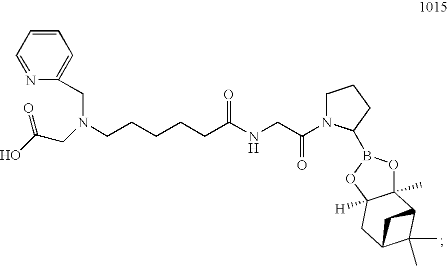

Re(CO)₃[2-((6-(2-(2-boronopyrrolidin-1-yl)-2-oxoethylamino)-6-oxohexyl)(pyridin-2-ylmethyl)amino)acetic acid] (1016), ESI MS m/z ESI MS m/z 726.0 (M+Na⁺); ¹H NMR (400 Hz, CD₃OD): δ 8.81 (d, J=13.0 Hz, 1H), 8.09 (m, 1H), 7.72 (m, 1H), 7.53 (m, 1H), 4.75 (m, 1H), 4.54 (m, 1H), 3.74 (s, 2H), 3.58 (m, 2H), 3.52 (m, 1H), 2.33 (m, 2H), 2.36 (m, 2H), 2.16 (bs, 1H), 1.94 (m, 4H), 1.78 (m, 2H), 1.67 (m, 1H), 1.76-1.94 (m, 4H), 1.46 (m, 2H), (amide NH's not seen):

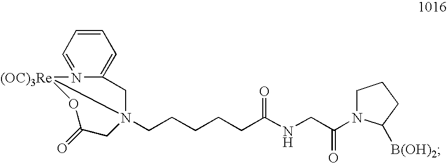

1-(2-(6-(bis((1-methyl-1H-imidazol-2-yl)methyl)amino)hexanamido) acetyl)pyrrolidin-2-ylpinadiol borate (1017), ESI MS m/z 304.0 (M/2+H⁺):

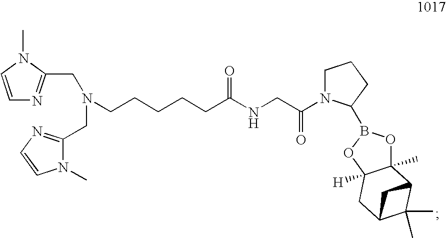

Re(CO)₃[1-(2-(6-(bis((1-methyl-1H-imidazol-2-yl)methyl)amino) hexanamido)acetyl)pyrrolidin-2-ylboronic acid] (1018), ESI MS m/z 744.0 (M+H⁺); ¹H NMR (400 Hz, CD₃OD): δ 7.98 (s, 1H), 7.09 (s, 2H), 7.05 (s, 2H), 4.66 (m, 2H), 4.60 (m, 2H), 3.99 (m, 3H), 3.72 (s, 6H), 3.55 (m, 2H), 3.31 (s, 2H), 2.45 (m, 1H), 2.36 (t, J=17.0 Hz, 2H), 2.16 (bs, 1H), 1.94 (m, 4H), 1.78 (m, 2H), 1.67 (m, 1H), 1.46 (m, 2H), 1.29 (s, 1H):

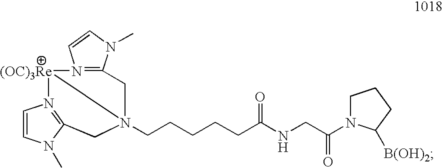

1-(2-(6-(bis((1-(2-tert-butoxy-2-oxoethyl)-1H-imidazol-2-yl)methyl)amino) hexanamido)acetyl)pyrrolidin-2-ylpinadiol borate (1019), ESI MS m/z 404.0 (M/2+H⁺):

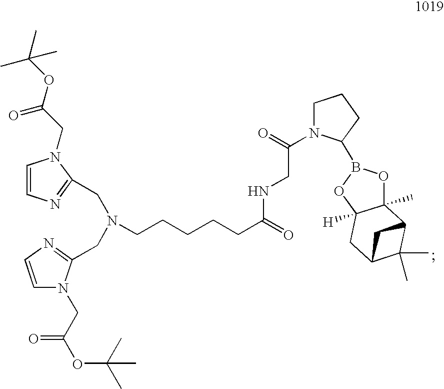

Re(CO)₃-2,2′-(2,2′-(6-(2-(2-boronopyrrolidin-1-yl)-2-oxoethylamino)-6-oxohexyl azanediyl)bis(methylene)bis(1H-imidazole-2,1-diyl))diacetic acid (1020), ESI MS m/z 417.0 (M/2+H⁺); ¹H NMR (400 Hz, CD₃OD): δ 7.96 (d, J=9.0 Hz, 4H), 4.81 (d, J=9.0 Hz, 4H), 4.33 (m, 4H), 3.84 (m, 2H), 3.62 (m, 2H), 3.45 (m, 2H), 2.96 (m, 1H), 2.24 (t, J=19.0 Hz, 2H), 2.05 (m, 1H), 1.93 (s, 1H), 1.76-1.90 (m, 4H), 1.55-1.68 (m, 4H), 1.32-1.38 (m, 2H), (CO₂H′ s not seen):

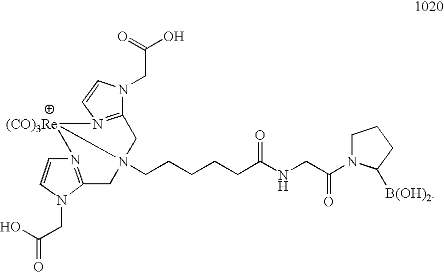

### Example 7

### Synthesis of Proline Diphenyl Phosphonate

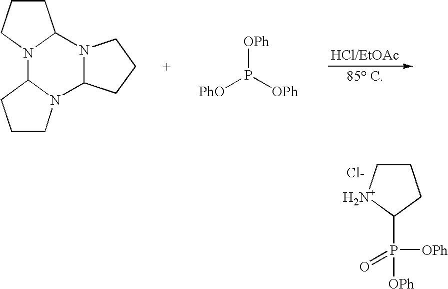

Proline diphenyl phosphonate can be synthesized followed the known procedure (Boduszek B. et al. J. Med. Chem. 1994, 37, 3969-3976; Nomura Y. et al. Chem. Lett. (Japan) 1977, 693-696) by reaction of triphenyl phosphate and 1-pyrroline trimer with HCl in EtOAc at 85° C.

### Example 8

### Synthesis of N-Protected pyrrolidine-2-carbonitrile

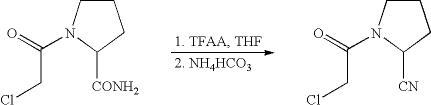

There are several procedures known in the art can be used to transform a carboxylic acid or amide group to a nitrile functional group. The example given here is by transformation of amide to nitrile. A solution of the amide in THF is treated with trifluoroacetic anhydride (TFAA). After completion of the reaction the side product can be neutralized by ammonium bicarbonate (NH₄HCO₃), and the nitrile can be isolated from the toluene extract without aqueous work up. The N-protected pyrrolidine-2-carbonitrile is then subject to deprotection to undergo further reaction at the ring nitrogen center such as reductive amination or peptide formation as described before.

### Example 9

### Preparation of Cyanoproline Complexes of Formula I

Following the similar chemistry as for preparation of the boronoproline derivatives, compound 1022 was prepared from commercially available pyrrolidine-2-carbonitrile.

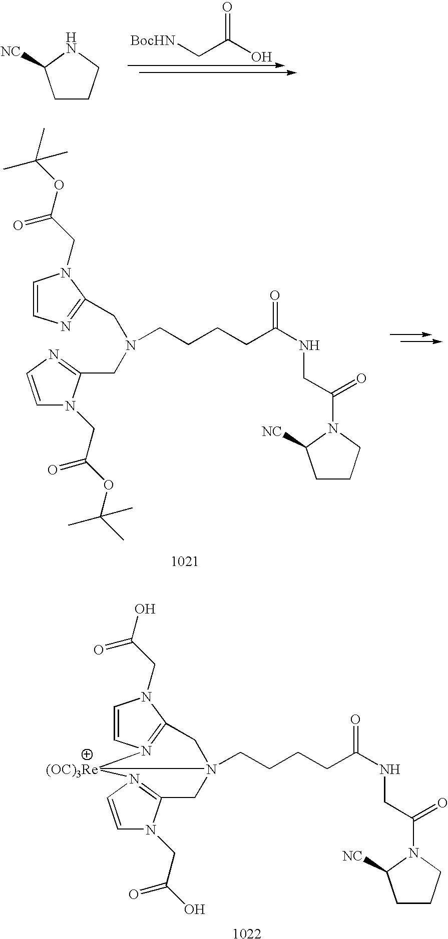

The compounds of the above synthetic scheme and their characterization data include, (S)-tert-butyl-2,2′-(2,2′-(6-(2-(2-cyanopyrrolidin-1-yl)-2-oxoethylamino)-6-oxohexylazanediyl) bis(methylene)bis(1H-imidazole-2,1-diyl))diacetate (1021), ESI MS m/z 417.0 (M/2+H⁺); and Re(CO)₃-(S)-2,2′-(2,2′-(6-(2-(2-cyanopyrrolidin-1-yl)-2-oxoethylamino)-6-oxohexylazanediyl)bis(methylene)bis(1H-imidazole-2,1-diyl))diacetic acid (1022) ESI MS m/z 814 (M+H⁺); ¹H NMR (400 Hz, CDCl₃): δ 7.07 (d, J=4.0 Hz, 2H), 7.05 (d, J=4.0 Hz, 2H), 4.83-4.91 (m, 6H), 4.38-4.51 (m, 4H), 4.03 (s, 2H), 3.74 (m, 2H), 3.67 (m, 2H), 3.60 (m, 2H), 2.36 (m, 2H), 2.24 (m, 1H), 2.17 (m, 1H), 1.91 (m, 2H), 1.75 (m, 2H), 1.44 (m, 2H), (CO₂H′ s not seen).

### Example 10

### Synthesis of Substituted Benzamide Boronoproline Derivatives

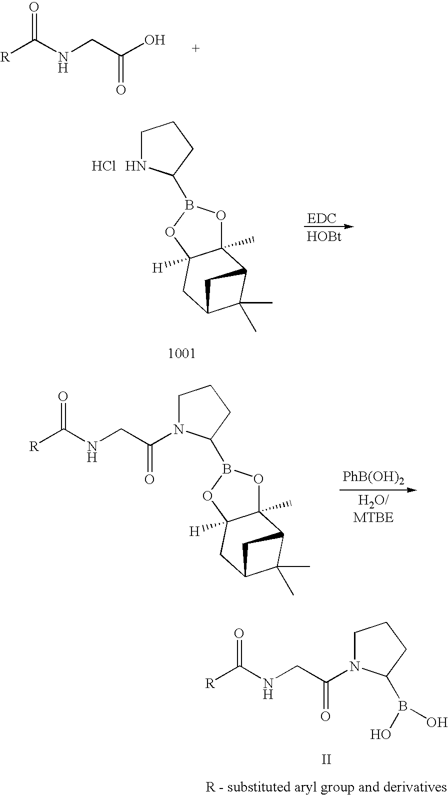

Proline boronic esters are prepared as described in the literature. The appropriately substituted benzamide-glycine is coupled to 1001 in the presence of 1-(3-(dimethylamino)propyl)-3-ethylcarodiimide hydrochloride (EDC) to form the protected boronic ester. Deprotection of the boronic ester was achieved by transesterfication with phenylboronic acid in a biphasic methyl tert-butyl ether (MTBE)-water mixture. The product, boronic acids of Formula II were isolated from the organic phase and purified by column chromatography or reverse phase HPLC.

General Method for Peptide Coupling. To a solution of the iodine substituted benzamido acetic acid (200.0 mg, 0.66 mmol) in CH₂Cl₂ (5.0 mL) was added hydroxybenzotriazole (89.0 mg, 0.66 mmol) and EDC (164.0 mg, 0.85 mmol). After 30 minutes, the pinanediol ester of proline boronic acid (1001, 187.0 mg, 17.5 mmol) and N-methylmorpholine (0.15 mL, 1.31 mmol) were added. After stirring overnight, the mixture was washed sequentially with water, 1 M KHSO₄, and Na₂CO₃ solutions. The organic layer was filtered through a plug of silica gel, eluting with EtOAc. Evaporation of the solvent yielded the protected dipeptides.

General Method for the Synthesis of Boronic Acid Dipeptides. A solution of the protected boronic esters (311.0 mg, 0.60 mmol) in H₂O (2.0 mL) was adjusted to pH=2 by addition of dilute HCl. Methyl tert-butyl ether (2.0 mL) and phenyl boric acid (78.0 mg, 0.64 mmol) were added, and the two-phase mixture was stirred vigorously. After continuing stirring overnight, the organic layer was separated and the solvent was removed. The desired product boronic acid II was obtained by reverse-phase HPLC purification.

Compounds prepared by the above general methods include: 1-(2-(2-iodobenzamido)acetyl)pyrrolidin-2-ylboronic acid (1023), ESI MS m/z 425 (M+Na⁺); ¹H NMR (400 Hz, CD₃OD): δ 7.81 (d, J=20.0 Hz, 1H), 7.37 (m, 2H), 7.07 (d, J=20.0 Hz, 1H), 4.52 (s, 2H), 4.06-4.52 (m, 2H), 3.63-3.41 (m, 3H), 2.15-1.81 (m, 4H), 1.65-1.52 (m, 2H):

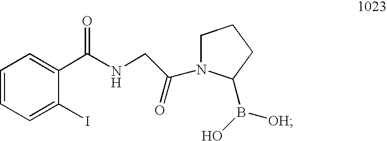

1-(2-(4-iodobenzamido)acetyl)pyrrolidin-2-ylboronic acid (1024), ESI MS m/z 425 (M+Na⁺); ¹H NMR (400 Hz, CD₃OD): δ 7.74 (d, J=22.0 Hz, 2H), 7.51 (d, J=22.0 Hz, 2H), 4.32-4.01 (m, 2H), 3.60-3.41 (m, 2H), 3.31-3.20 (m, 1H), 2.15-1.80 (m, 4H), 1.65-1.55 (m, 2H):

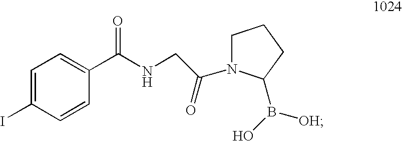

1-(2-(3-iodobenzamido)acetyl)pyrrolidin-2-ylboronic acid (1025), ESI MS m/z 425 (M+Na⁺); NMR (400 Hz, CD₃OD): δ 8.14 (s, 1H), 7.8 (m, 2H), 7.15 (m, 1H), 4.32-4.01 (m, 2H), 3.62-3.41 (m, 2H), 3.0 (m, 1H), 2.15-1.80 (m, 4H), 1.73-1.50 (m, 2H):

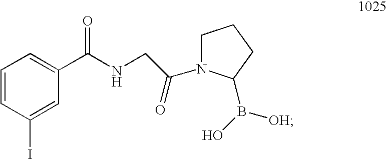

1-(2-(2-chloro-4-iodobenzamido)acetyl)pyrrolidin-2-ylboronic acid (1026) ESI MS m/z 459 (M+Na⁺); ¹H NMR (400 Hz, CD₃OD): δ 7.88 (s, 1H), 7.77 (d, J=20.0 Hz, 2H), 7.36 (d, J=20.0 Hz, 2H), 4.32-4.11 (m, 2H), 3.60-3.41 (m, 4H), 3.22 (m, 1H), 2.18 (m, 1H), 2.00 (m, 2H), 1.72 (m, 1H), 1.38 (m, 1H):

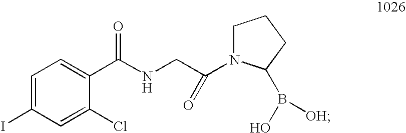

1-(2-(2-chloro-5-iodobenzamido)acetyl)pyrrolidin-2-ylboronic acid (1027), ESI MS m/z 459 (M+Na⁺); NMR (400 Hz, CD₃OD): δ 7.98 (s, 1H), 7.79 (m, 2H), 7.25 (m, 2H), 4.18 (m, 2H), 3.65 (m, 1H), 3.58 (m, 2H), 3.22 (m, 1H), 2.18 (m, 1H), 2.02 (m, 3H), 1.72 (m, 1H), 1.30 (m, 1H):

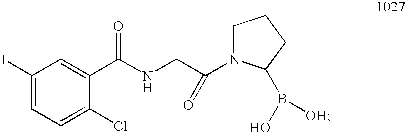

1-(2-(2-bromo-5-iodobenzamido)acetyl)pyrrolidin-2-ylboronic acid (1028), ESI MS m/z 503.0 (M+Na⁺); NMR (400 Hz, CD₃OD): δ 7.91 (d, J=5.0 Hz, 1H), 7.67 (dd, J=20.0, 5.0 Hz, 1H), 7.39 (dd, J=20.0, 5.0 Hz, 1H), 4.17 (m, 2H), 3.63 (m, 1H), 3.53 (m, 1H), 3.13 (m, 1H), 2.16 (m, 2H), 2.02 (m, 3H), 1.71 (m, 1H), (amide NH's not seen):

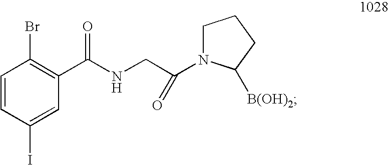

1-(2-(2-fluoro-5-iodobenzamido)acetyl)pyrrolidin-2-ylboronic acid (1029), ESI MS m/z 443.0 (M+Na⁺); ¹H NMR (400 Hz, CD₃OD): δ 8.18 (m, 1H), 7.84 (m, 1H), 7.33 (m, 1H), 7.01 (m, 1H), 4.17 (m, 2H), 3.64 (m, 1H), 3.52 (m, 1H), 3.13 (m, 1H), 2.17 (m, 1H), 2.04 (m, 3H), 1.72 (m, 2H):

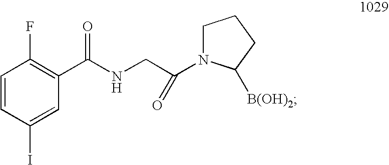

1-(2-(6-iodo-2-naphthamido)acetyl)pyrrolidin-2-ylboronic acid (1030), ESI MS m/z 475.0 (M+Na⁺); ¹H NMR (400 Hz, CD₃OD): δ 8.29 (d, J=17.0 Hz, 1H), 7.99 (dd, J=20.0, 9.0 Hz, 2H), 7.76 (d, J=17.0 Hz, 1H), 7.56 (m, 1H), 7.22 (m, 1H), 4.30 (m, 2H), 3.70 (m, 3H), 3.11 (m, 1H), 2.21 (m, 1H), 2.09-1.95 (m, 2H), 1.72 (m, 1H), 1.30 (bs, 1H), (amide NH's not seen):

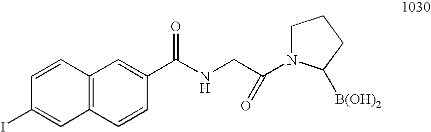

1-(2-(2-cyano-5-iodobenzamido)acetyl)pyrrolidin-2-ylboronic acid (1031), ESI MS m/z 450 (M+Na⁺); NMR (400 Hz, CD₃OD): δ 8.07 (d, J=20.0 Hz, 1H), 7.72 (bs, 1H), 7.57 (d, J=20.0 Hz, 1H), 7.36 (m, 1H), 4.10-4.27 (m, 2H), 3.31-3.68 (m, 4H), 3.12 (m, 1H), 2.17 (m, 1H), 2.01 (m, 2H), 1.71 (m, 1H):

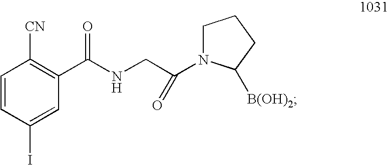

1-(2-(5-iodo-2-methylbenzamido)acetyl)pyrrolidin-2-ylboronic acid (1032), ESI MS m/z 439 (M+Na⁺); ¹H NMR (400 Hz, CD₃OD): δ 7.82 (d, J=4.0 Hz, 1H), 7.68 (dd, J=20.0, 4.0 Hz, 1H), 7.05 (d, J=20.0 Hz, 1H), 4.10-4.27 (m, 2H), 3.52-3.66 (m, 2H), 3.11 (m, 1H), 2.36 (s, 3H), 2.18 (m, 1H), 2.03 (m, 3H), 1.70 (m, 2H), (amide NH's not seen):

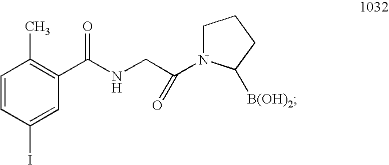

1-(2-(4-iodopicolinamido)acetyl)pyrrolidin-2-ylboronic acid (1033), ESI MS m/z 503.0 (M+Na⁺); NMR (400 Hz, CD₃OD): δ 8.69 (s, 1H), 8.54 (s, 1H), 8.50 (m, 1H), 8.37 (m, 1H), 4.20-4.31 (m, 2H), 3.44 (m, 2H), 2.45 (m, 1H), 1.93-2.05 (m, 4H), 1.71 (m, 2H).

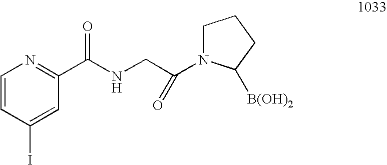

1-(2-(3-(4-iodophenyl)ureido)acetyl)pyrrolidin-2-ylboronic acid (1034), prepared under the similar condition as in as in the general method for peptide coupling, above, using 2-(3-(4-iodophenyl)ureido)acetic acid instead of benzamido acetic acid, ESI MS m/z 418 (M+H⁺); NMR (400 Hz, CD₃OD): δ 7.55 (d, J=22.0 Hz, 2H), 7.21 (d, J=22.0 Hz, 2H), 4.00-4.09 (m, 2H), 3.68 (m, 1H), 3.58 (m, 1H), 3.50 (m, 1H), 3.10 (m, 1H), 2.16 (m, 1H), 1.94-2.05 (m, 2H), 1.68 (m, 1H), 1.29-1.37 (m, 1H), (urea NH's not seen):

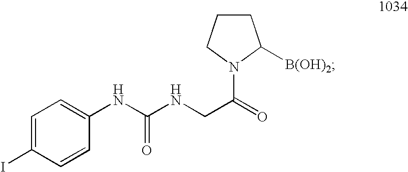

1-(2-(2-amino-3-(4-iodophenyl)propanamido)acetyl)pyrrolidin-2-ylboronic acid (1035), ESI MS m/z 428 (M⁺−OH); ¹H NMR (400 Hz, CD₃OD): δ 7.71 (d, J=21.0 Hz, 2H), 7.07 (d, J=21.0 Hz, 2H), 4.12 (m, 3H), 3.85 (m, 1H), 3.68 (m, 2H), 3.52 (m, 2H), 3.44 (m, 2H), 3.01-3.25 (m, 2H), 2.16 (bs, 2H), 1.99 (m, 2H), 1.69 (m, 1H):

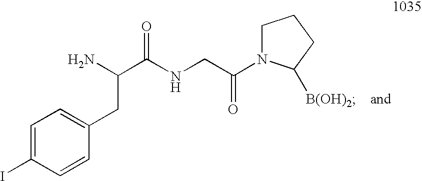

1-(2-(4-(3-iodobenzyl)piperazin-1-yl)acetyl)pyrrolidin-2-ylboronic acid (1036), prepared under the similar condition as in as in the general method for peptide coupling, above, using 2-(4-(3-iodobenzyl)piperazin-1-yl)acetic acid instead of benzamido acetic acid, ESI MS m/z 479.0 (M+Na⁺); ¹H NMR (400 Hz, CD₃OD): δ 7.86 (s, 1H), 7.77 (d, J=20.0 Hz, 1H), 7.44 (d, J=20.0 Hz, 1H), 7.24 (m, 1H), 3.98 (s, 2H), 3.76 (s, 2H), 3.53 (m, 2H), 3.45 (m, 2H), 3.11 (bs, 9H), 2.13 (m, 1H), 2.02 (m, 2H), 1.67 (m, 1H):

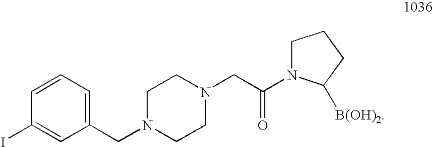

### Example 11

### Synthesis of the Trimethylstannyl Precursor to the Radiolabeled Compound 1039

Radiolabeled 1039, or the like, may be prepared via a trimethylstannyl precursor followed by radioiododestannylation. Compound 1037 was prepared and then coupled with boronoproline 1007 to afford the trimethylstannyl precursor 1038:

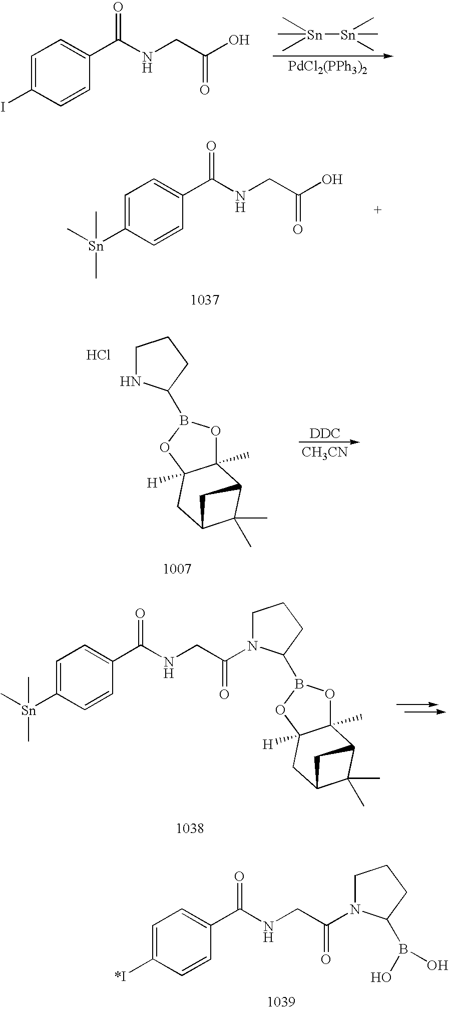

As outlined above, 2-(4-(trimethylstannyl)benzamido)acetic acid 1037, may be prepared via the following. To a solution of 2-(4-iodobenzamido)acetic acid (262.0 mg, 0.86 mmol) in dry dioxane (5.0 mL) was added hexamethylditin (702 mg, 2.14 mmol) followed by Pd(Ph₃P)₂Cl₂ (120.0 mg, 0.04 mmol), and the reaction mixture was heated for 3 h under reflux. The mixture was filtered through Celite and purified by column chromatography (SiO₂) using hexanes/ethyl acetate (9/1) as eluent to afford 1037 as clear oil. ESI MS m/z 344.0 (M+Hⁱ).

1-(2-(4-(trimethylstannyl)benzamido)acetyl)pyrrolidin-2-yl-pinanediol boronate (1038), was also prepared and characterized: ESI MS m/z 574 (M+H⁺); ¹H NMR (400 Hz, CDCl₃): 7.77 (d, J=19.0 Hz, 2H), 7.55 (d, J=19.0 Hz, 2H), 4.35 (m, 1H), 4.17 (m, 2H), 3.49 (m, 2H), 3.20 (m, 1H), 2.31 (m, 1H), 2.00-2.21 (m, 5H), 1.88 (m, 4H), 1.97 (s, 1H), 1.30 (s, 3H), 0.84 (s, 6H), 0.30 (s, 9H).

### Example 12

### Synthesis of Substituted Benzamide Cyanoproline Derivatives

Under the similar chemistry as describe before, by employing various cyanoprolines as starting materials, several substituted benzamide cyanoproline derivatives were prepared.

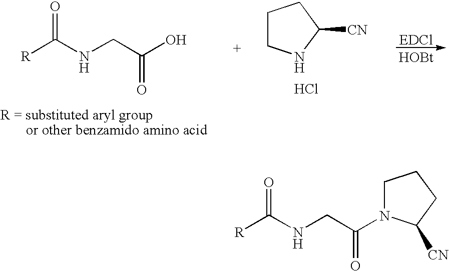

General Method for Peptide Coupling. To a solution of the iodine substituted benzamido glycine (or other benzamido amino acid) (200.0 mg, 0.66 mmol) in CH₂Cl₂ (5.0 mL) was added hydroxybenzotriazole (89.0 mg, 0.66 mmol) and EDC (164.0 mg, 0.85 mmol). After 30 min, cyanoproline (187.0 mg, 17.5 mmol) and N-methylmorpholine (0.15 mL, 1.31 mmol) were added. After stirring overnight, the mixture was washed sequentially with water, 1 M KHSO₄, and Na₂CO₃ solutions. The organic layer was filtered through a plug of silica gel, eluting with EtOAc. Evaporation of the solvent yielded the crude product which was purified by HPLC purification to afford the pure desired products. Products of this method and their characterization data include: (S)-N-(2-(2-cyanopyrrolidin-1-yl)-2-oxoethyl)-4-iodobenzamide 1039), ESI MS m/z 384.0 (M+H⁺); NMR (400 Hz, CDCl₃): δ 7.93 (bs, 1H), 7.74 (d, J=20.0 Hz, 2H), 7.46 (d, J=20.0 Hz, 2H), 4.72 (m, 1H), 4.52 (m, 2H), 4.03 (m, 1H), 3.69 (bs, 1H), 3.47 (m, 1H), 2.21 (m, 3H):

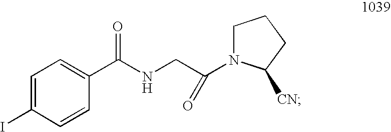

(S)-N-(2-(2-cyanopyrrolidin-1-yl)-2-oxoethyl)-3-iodobenzamide (1040), ESI MS m/z 384.0 (M+H⁺); ¹H NMR (400 Hz, CDCl₃): δ 8.15 (s, 1H), 7.76 (m, 2H), 7.56 (m, 1H), 7.09 (m, 1H), 4.76 (d, J=17.0 Hz, 1H), 4.13-4.43 (m, 2H), 3.69 (m, 1H), 3.51 (m, 1H), 2.16 (m, 4H):

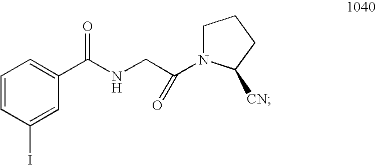

(S)-2-chloro-N-(2-(2-cyanopyrrolidin-1-yl)-2-oxoethyl)-4-iodobenzamide (1041), ESI MS m/z 418.0 (M+H⁺); ¹H NMR (400 Hz, CDCl₃): δ 7.81 (m, 1H), 7.68 (d, J=21.0 Hz, 1H), 7.45 (bs, 1H), 7.41 (d, J=21.0 Hz, 1H), 4.78 (m, 1H), 4.17-4.36 (m, 2H), 3.72 (m, 1H), 3.51 (m, 1H), 2.21-2.38 (m, 4H):

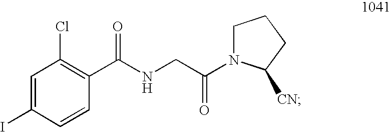

(S)-2-chloro-N-(2-(2-cyanopyrrolidin-1-yl)-2-oxoethyl)-5-iodobenzamide (1042), ESI MS m/z 418.0 (M+H⁺); ¹H NMR (400 Hz, CDCl₃): δ 7.98 (m, 1H), 7.69 (d, J=21.0, Hz, 1H), 7.36 (bs, 1H), 7.15 (d, J=21.0 Hz, 1H), 4.78 (m, 1H), 4.17-4.36 (m, 2H), 3.71 (m, 1H), 3.51 (m, 1H), 2.20-2.37 (m, 4H):

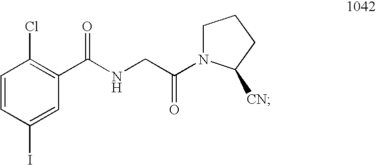

(S)—N-(2-(2-cyanopyrrolidin-1-yl)-2-oxoethyl)-2-iodobenzamide (1043), ESI MS m/z 384.0 (M+H⁺); ¹H NMR (400 Hz, CDCl₃): δ 7.88 (d, J=20.0 Hz, 1H), 7.41 (m, 2H), 7.13 (d, J=20.0 Hz, 1H), 6.99 (bs, 1H), 4.78 (m, 1H), 4.19-4.37 (m, 2H), 3.72 (m, 1H), 3.52 (m, 1H), 2.21-2.37 (m, 4H);

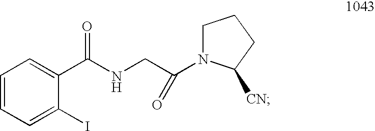

(S)-2-cyano-N-(2-(2-cyanopyrrolidin-1-yl)-2-oxoethyl)-5-iodobenzamide (1044), ESI MS m/z 432.0 (M+Na⁺); ¹H NMR (400 Hz, CDCl₃): δ 7.98 (d, J=4.0 Hz, 1H), 7.69 (d, J=12.0 Hz, 1H), 7.50 (bs, 1H), 7.15 (d, J=12.0 Hz, 1H), 4.78 (m, 1H), 4.19-4.48 (m, 2H), 3.75 (m, 1H), 3.56 (m, 1H), 2.23-2.35 (m, 4H):

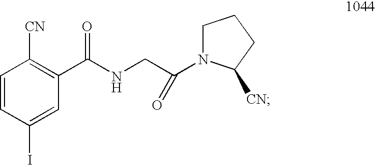

(S)-3-(2-(2-cyanopyrrolidin-1-yl)-2-oxoethylcarbamoyl)-5-iodobenzoic acid (1045), ESI MS m/z 428.0 (M+H⁺); ¹H NMR (400 Hz, CDCl₃): δ 8.50 (m, 2H), 8.43 (s, 1H), 4.22 (m, 2H), 3.77 (m, 2H), 3.63 (m, 2H), 2.21-2.26 (m, 4H), (CO₂H's not seen):

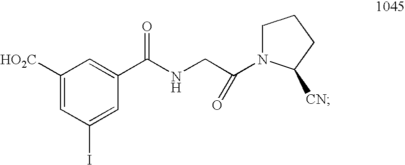

(R)-N-(2-(2-cyanopyrrolidin-1-yl)-2-oxoethyl)-3-iodobenzamide (1046), ESI MS m/z 384.0 (M+H⁺); ¹H NMR (400 Hz, CDCl₃): δ 8.15 (s, 1H), 7.76 (m, 2H), 7.52 (bs, 1H), 7.09 (m, 1H), 4.77 (m, 1H), 4.41 (m, 1H), 4.10 (m, 1H)), 3.69 (m, 1H), 3.51 (m, 1H), 2.14-2.36 (m, 4H):

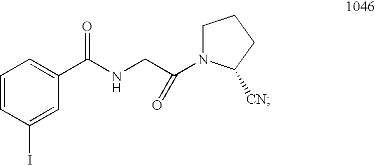

(S)-N-(2-(2-cyanopyrrolidin-1-yl)-2-oxoethyl)-3-(trimethylstannyl)benzamide (1047), ESI MS m/z 42.0 (M+H¹); ¹H NMR (400 Hz, CDCl₃): δ 7.94 (s, 1H), 7.74 (d, J=20.0 Hz, 1H), 7.67 (m, 1H), 7.54 (m, 1H), 7.79 (m, 1H), 4.34 (m, 1H), 4.18 (m, 1H), 3.69 (m, 1H), 3.55 (m, 1H), 2.34 (m, 2H), 2.25 (m, 3H), 0.32 (s, 9H):

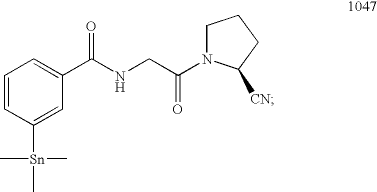

(R)-4-(2-((S)-2-cyanopyrrolidin-1-yl)-2-oxoethylamino)-3-(3-iodobenzamido)-4-oxobutanoic acid (1048), prepared under the similar condition as in the general method for peptide coupling, above, using 3-iodobenzoic acid, Asp-Gly and cyanoproline, ESI MS m/z 499.0 (M+H⁺); ¹H NMR (400 Hz, CDCl₃): δ 8.20 (s, 1H), 7.90 (d, J=20.0 Hz, 1H), 7.82 (d, J=20.0 Hz, 1H), 7.24 (m, 1H), 5.36 (m, 1H), 4.76 (m, 1H), 4.06 (m, 1H)), 3.87 (m, 1H), 3.51-3.71 (m, 3H), 3.22 (m, 1H), 2.95-3.03 (m, 2H), 2.74 (m, 1H), 2.14-2.69 (m, 2H). (amide NH and CO₂H are not seen):

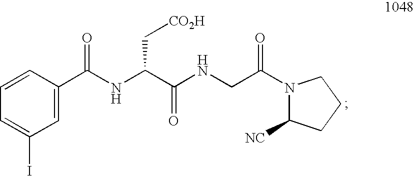

(S)-1-(2-(3-iodobenzylamino)acetyl)pyrrolidine-2-carbonitrile (1049), The title compound was prepared under the similar condition as in the general method for peptide coupling, above, using 2-(3-iodobenzylamino)acetic acid instead of benzamido glycine, ESI MS m/z 370.0 (M+H⁺); ¹H NMR (400 Hz, CDCl₃): δ 7.94 (s, 1H), 7.84 (d, J=20.0 Hz, 1H), 7.52 (d, J=18.0 Hz, 1H), 7.26 (m, 1H), 4.76 (m, 1H), 4.06 (m, 2H), 3.88 (m, 2H), 3.68 (m, 2H), 3.45 (m, 1H), 2.06-2.36 (m, 4H):

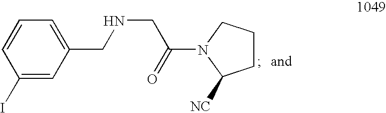

(S)-1-(2-(4-(3-iodobenzyl)piperazin-1-yl)acetyl)pyrrolidine-2-carbonitrile (1050), The title compound was prepared under the similar condition as in as in the general method for peptide coupling, above, using 2-(4-(3-iodobenzyl)piperazin-1-yl)acetic acid instead of benzamido glycine, ESI MS m/z 439.0 (M+H⁺); ¹H NMR (400 Hz, CDCl₃): δ 7.89 (s, 1H), 7.82 (d, J=15.0 Hz, 1H), 7.52 (d, J=20.0 Hz, 1H), 7.24 (m, 1H), 4.78 (m, 1H), 3.93 (s, 2H), 3.75 (s, 2H), 3.53 (m, 1H), 3.45 (m, 1H), 3.11 (bs, 8H), 2.13 (d, J=16 Hz, 1H), 2.02 (m, 2H), 1.67 (m, 1H):

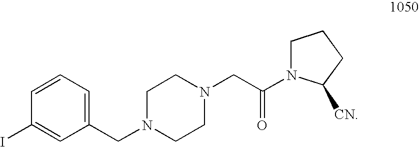

### Example 13

### General Radiolabeling Procedure

[^{99m}Tc(CO)₃(H₂O)₃]⁺can be prepared by the methods known in the art using the Isolink® radiolabeling kits available from Tyco Healthcare, St. Louis, Mo. Sodium Pertechnetate, 7400 MBq (200 mCi), in saline (2.5 mL) is added to an Isolink® radiolabeling kit and the vial is placed in an oil bath at 100° C. The reaction will be heated for 45 minutes and 1N HCl (200 μL) be then added to neutralize the reaction mixture. The product, [^{99m}Tc(CO)₃(H₂O)₃]⁺, is removed from the vial via syringe and added to another vial containing compounds with chelates (200 μL of a 1 mg/mL solution in methanol) followed by an additional amount of methanol (0.3 mL). The reaction is heated for 1 hour at 80° C. and the crude reaction is injected on the HPLC to determine radiochemical yield (RCY). Exemplary Compound 1014Tc (the Tc-99m analog of the rhenium-based compound 1014) was prepared accordingly at scale of 25 mCi with 71% radiochemical yield (RCY) in >98% radiochemical purity (RCP) after reverse phase-HPLC purification. Stability was evaluated at room temperature by quantitation of free Compound 1014Tc content present by HPLC. As shown in FIG. 4, 97% radiochemical purity (RCP) remained after 5 hours.

### Example 14

### General Procedure for the Preparation of Iodine-123 and Iodine-131 Analogs

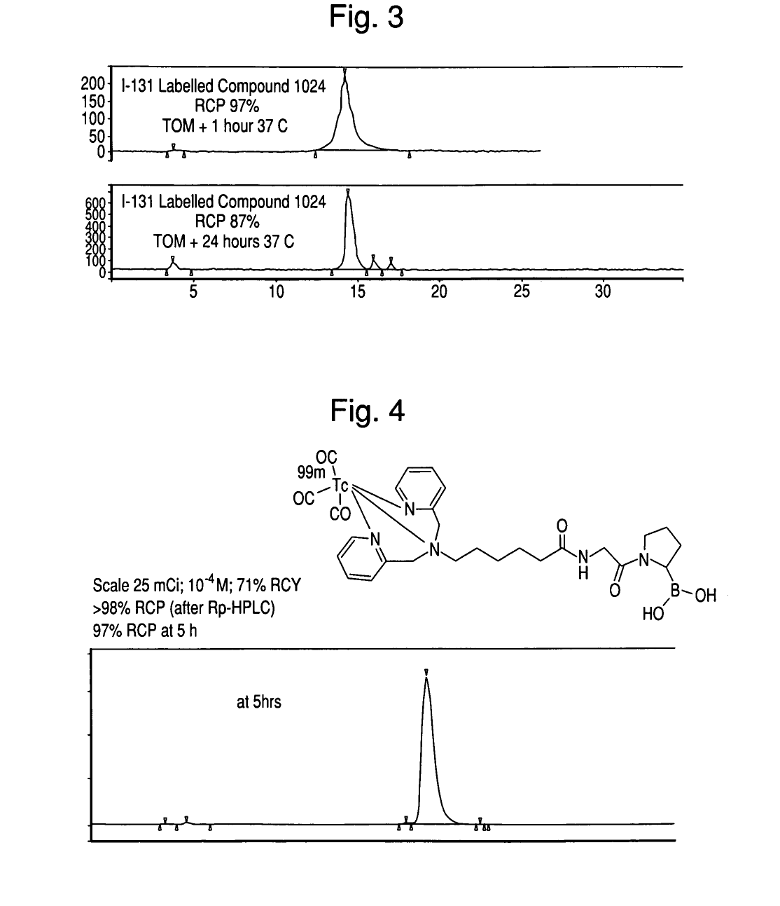

In a 5 ml vial containing Na*I (*I-123 or *I-131; 2-300 mCi) was added sterile water for injection (SWFI) (50 μL), 50% sulfuric acid in SWFI (50 μl), oxidant (100 μL) [which was prepared fresh via the incubation of acetic acid (0.2 mL) and 30% hydrogen peroxide (0.335 mL) followed by dilution to a final volume of 5 mL with SWFI], acetonitrile (0.5 mL), and the trimethylstannyl-precursor (100 μL of a 1 mg/mL solution in acetonitrile). The mixture was vortexed for 1 min and allowed to incubate at room temperature for an additional 10 minutes. The reaction was quenched with 200 μL of 0.1 M sodium thiosulfate. The product was then purified using Rp-HPLC employing a C18 column with a gradient HPLC method with acetonitrile+0.1% TFA as the eluting solvent. The solution was evaporated to dryness under a stream of nitrogen and the residue was dissolved in a formulation matrix of 10% ethanol in saline. The radiochemical yields ranged from 50-70%, RCP >90% specific activity ≧4000 mCi/μmol. Exemplary I-131 labelled compound 1024 was prepared accordingly. A radiochromatogram of the HPLC purified 1-131 labelled compound 1024 is presented in FIG. 2 in comparison to the non-radiolabelled compound 1024 as an identity standard. Stability was evaluated at 37° C. by quantitation of free 1-131 labelled compound 1024 content present by HPLC. The result (FIG. 3), demonstrated that 87% radiochemical purity (RCP) remained at time of manufacture (TOM) plus 24 hours.

### Example 15

### In Vitro Screening

In vitro assaying of compounds for inhibition of dipeptidyl peptidase activity of seprase and DPP-IV can be achieved following the described in Edosada C. Y. et al. J. Biolog. Chem. 2006, 281, 7437-7444.

Selected test compounds were examined for their ability to inhibit the enzymatic activity of recombinant human seprase (rhFAP) on the substrate, benzyloxycarbonyl-Gly-Pro-7-amido-4-methylcoumarin (Z-Gly-Pro-AMC). In brief, 10 μg rhFAP was added to 50 μM Z-Gly-Pro-AMC in the presence of increasing concentrations of test compound, and enzymatic activity was determined by monitoring fluorescence (Ex. 355 nm/Em. 460 nm). Compound 1051 is the known inhibitor, Cbz-Gly-boroproline. Initial velocity of the reaction was calculated and normalized to control reactions conducted in the absence of test compound. The results for no inhibitor, and compounds 1010, 1023-1025, and 1051 are presented in Table 1.

TABLE 1
Percent of Control v. Concentration of Inhibitor

| Conc. | No | Cmpd. | Cmpd. | Cmpd. | Cmpd. | Cmpd. |
|---|---|---|---|---|---|---|
| (μM) | Inhibitor | 1023 | 1024 | 1025 | 1051 | 1010 |
| 0.005 | 91 | 42 | 22 | 0 | 81 | 90 |
| 0.05 | 94 | 0 | 0 | 0 | 46 | 95 |
| 0.5 | 100 | 0 | 0 | 0 | 1 | 82 |
| 5 | 100 | 0 | 0 | 0 | 0 | 26 |
| 50 | 96 | 0 | 0 | 0 | 0 | 0 |
| 500 | 91 | 0 | 0 | 0 | 0 | 0 |

Summary of in vitro data of exemplary compounds against seprase (fibroblast activation protein, FAP) is listed in Table 2. The inhibition was measured by the half maximal inhibitory concentration (IC₅₀) of a compound of interest. The IC₅₀ indicates how much of a particular compound is needed to inhibit seprase by half. Several compounds have IC₅₀ values in the low nanomolar range, e.g. 1024, 1040, and 1030.

TABLE 2
Summary of in vitro data against seprase

| Compound No. | FAP (IC₅₀ nM) |
|---|---|
| 1010 | 2,540 |
| 1014 | 21 |
| 1016 | 3,533 |
| 1018 | 20 |
| 1020 | 4 |
| 1022 | 236 |
| 1023 | 7 |
| 1024 | 3 |
| 1025 | 2 |
| 1026 | 3 |
| 1027 | 5 |
| 1028 | 5 |
| 1029 | 8 |
| 1030 | 2 |
| 1031 | 262 |
| 1032 | 11 |
| 1033 | 500 |
| 1034 | 11 |
| 1035 | 340 |
| 1036 | 37 |
| 1039 | 511 |
| 1040 | 35 |
| 1041 | 692 |
| 1042 | 785 |
| 1043 | 115 |
| 1044 | 23,680 |
| 1045 | 1,437 |
| 1046 | 970 |
| 1048 | 7,414 |
| 1049 | 953 |
| 1050 | 1152 |
| 1051 | 67 |
| 1060 | 41,300 |
| 1061 | 24,540 |

In Table 2, compounds 1060 and 1061 correspond to compounds of the following structures:

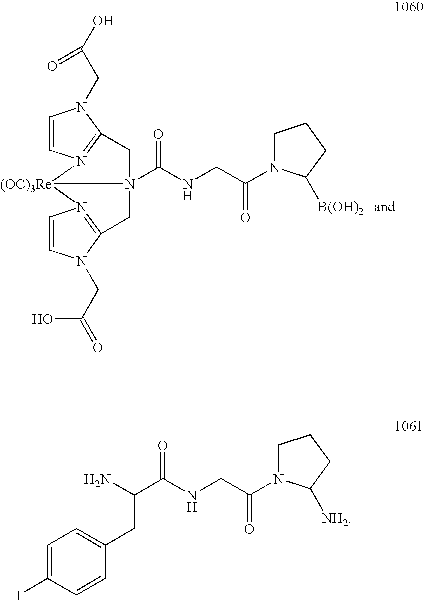

### Example 16

### Cell Based Enzyme Assay

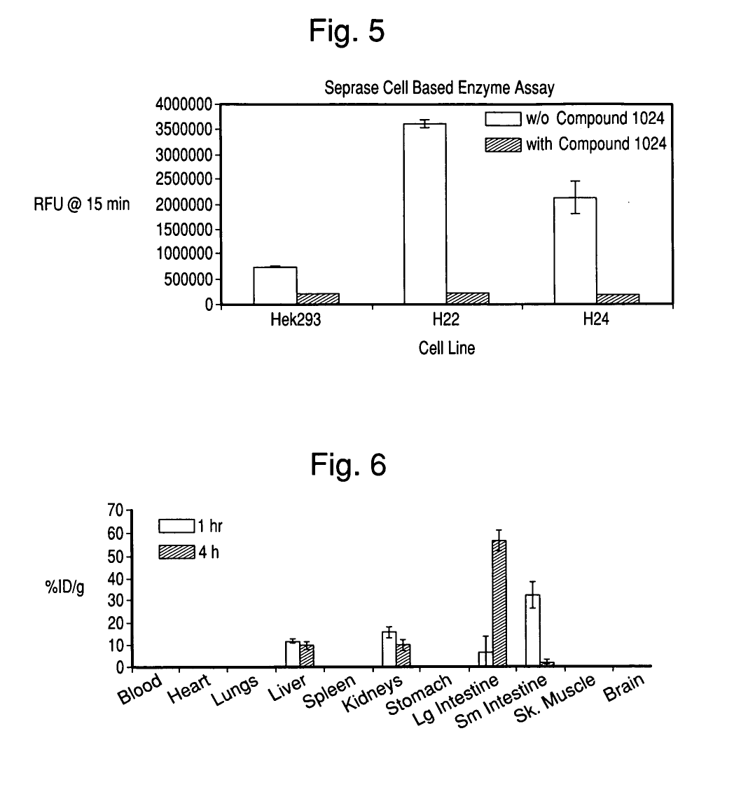

Cell based seprase inhibition was conducted with and without compound 1024. HEK293, H22 and H24 cells were incubated for 15 minute with and without about 25 mM of compound 1024, following the standard procedure known in the art. Fluorescence was measured at 15 minute to determine inhibition. The results are shown in FIG. 5.

### Mouse Tissue Distribution Studies.

A quantitative analysis of the tissue distribution of radiolabeled compounds was performed in separate groups of male normal mice or male NCr Nude−/− mice bearing seprase expressing FaDu or H22(+) xenografts (approximately 100-200 mm³) administered via the tail vein as a bolus injection (approximately 2 μCi/mouse) in a constant volume of 0.05 ml. The animals (n=5/time point) were euthanized by asphyxiation with carbon dioxide at 0.25, 1, 2, 4, 8, and 24 hours post injection. Tissues (blood, heart, lungs, liver, spleen, kidneys, adrenals, stomach, large and small intestines (with contents), testes, skeletal muscle, bone, brain, adipose, and tumor) were dissected, excised, weighed wet, transferred to plastic tubes and counted in an automated γ-counter (LKB Model 1282, Wallac Oy, Finland).

### Example 17

### Biodistribution Evaluation of Compound 1109

Tissue distribution data generated with a ^{99m}Tc complex, Compound 1014/1109, in normal mice, demonstrated greater small intestine uptake at one hour and greater large intestine uptake at 4 hours (FIG. 6).

### Example 18

### Biodistribution Evaluation of Compounds 1018 and 1110

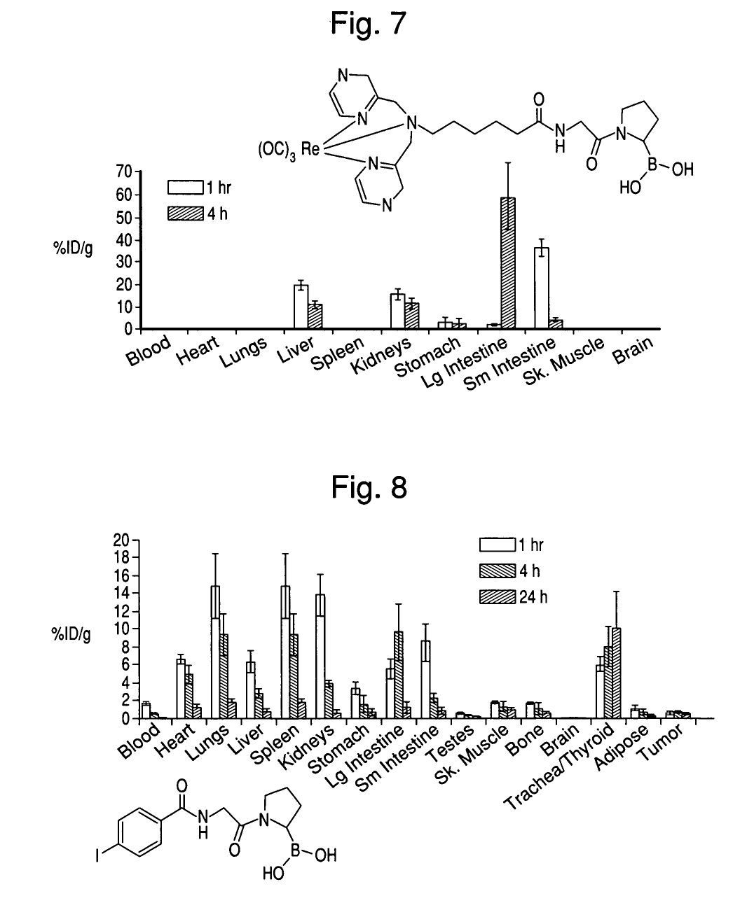

Tissue distribution data generated with Compound 1018/1110 in normal, mice demonstrated greater small intestine uptake at one hour and greater large intestine uptake at 4 hours (FIG. 7).

### Example 19

### Biodistribution Evaluation of 1-123 Labelled 1024 in FaDu Xenograft Mice

Tissue distribution data generated with 1-123 labelled 1024 in FaDu Xenograft rats are presented in FIG. 8. The data were generated at 1 hour, 4 hours and 24 hours.

### Example 20

### Biodistribution Evaluation of 1-123 Labelled Compound 1024 in H22(+) Xenograft Mice

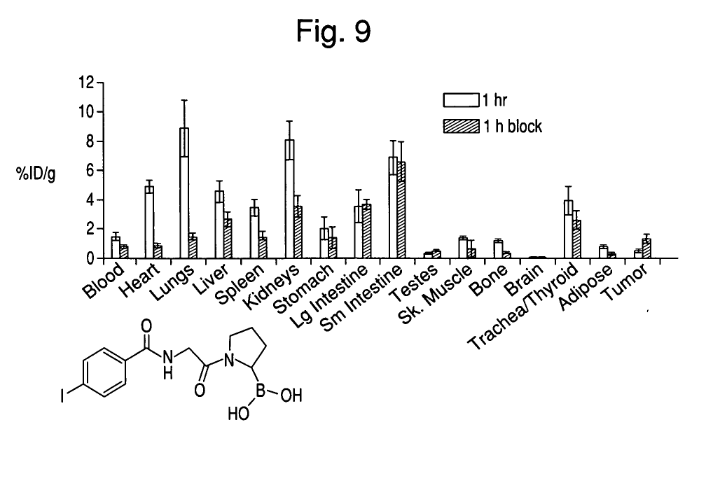

Tissue distribution data generated with 1-123 labelled compound 1024 in H22(+) Xenograft rats are presented in FIG. 9. The data were generated at 1 hour with blocking.

The following table (Table 3) is a listing of exemplary Seprase inhibitor compounds may generally be made using the methods described above. At the position of U, both R and S enantiomers are contemplated, even where enantiomeric designation is or is not provided. It is expected that these compounds will exhibit properties and activities similar to those exemplified above.

TABLE 3
Exemplary Seprase Inhibitors

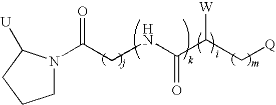

| Compound | | | | | | | |
|---|---|---|---|---|---|---|---|
| No. | U | j | k | i | m | W | Q |
| 1010 | B(OH)₂ | 1 | 0 | 0 | 4 | — | 1M |
| | | | | | | | M = Re |
| 1011 | B(OH)₂ | 1 | 1 | 0 | 5 | — | 9M |
| | | | | | | | M = Re |
| 1014 | B(OH)₂ | 1 | 1 | 0 | 5 | — | 1M |
| | | | | | | | M = Re |
| 1016 | B(OH)₂ | 1 | 1 | 0 | 5 | — | 3M |
| | | | | | | | M = Re |
| 1018 | B(OH)₂ | 1 | 1 | 0 | 5 | — | 7M |
| | | | | | | | M = Re |
| 1020 | B(OH)₂ | 1 | 1 | 0 | 5 | — | 8M |
| | | | | | | | M = Re |
| 1021 | (S)-CN | 1 | 1 | 0 | 5 | — | 8 |
| 1022 | (S)-CN | 1 | 1 | 0 | 5 | — | 8M |
| | | | | | | | M = Re |
| 1023 | B(OH)₂ | 1 | 0 | 0 | 0 | — | 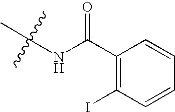 |
| 1024 | B(OH)₂ | 1 | 0 | 0 | 0 | — | 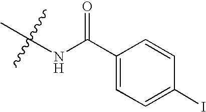 |
| 1025 | B(OH)₂ | 1 | 0 | 0 | 0 | — | 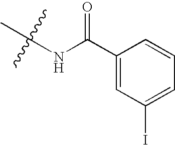 |
| 1026 | B(OH)₂ | 1 | 0 | 0 | 0 | — | 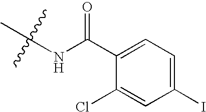 |
| 1027 | B(OH)₂ | 1 | 0 | 0 | 0 | — | 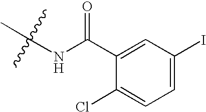 |
| 1028 | B(OH)₂ | 1 | 0 | 0 | 0 | — | 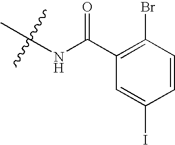 |
| 1029 | B(OH)₂ | 1 | 0 | 0 | 0 | — | 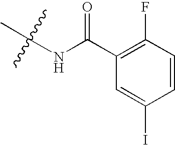 |
| 1030 | B(OH)₂ | 1 | 0 | 0 | 0 | — | 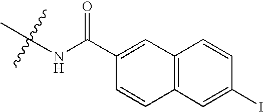 |
| 1031 | B(OH)₂ | 1 | 0 | 0 | 0 | — | 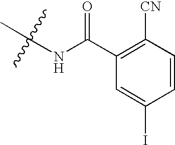 |
| 1032 | B(OH)₂ | 1 | 0 | 0 | 0 | — | 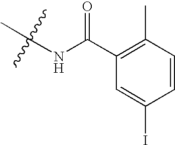 |
| 1033 | B(OH)₂ | 1 | 0 | 0 | 0 | — | 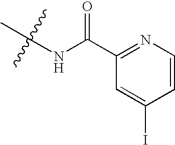 |
| 1034 | B(OH)₂ | 1 | 0 | 0 | 0 | — | 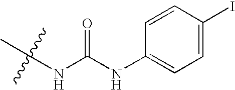 |
| 1035 | B(OH)₂ | 1 | 0 | 0 | 0 | — | 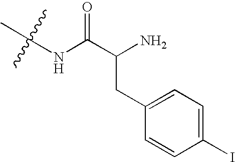 |
| 1036 | B(OH)₂ | 1 | 0 | 0 | 0 | — | 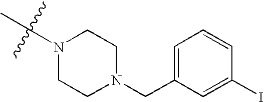 |
| 1037 | B(OR)₂ | 1 | 0 | 0 | 0 | — | 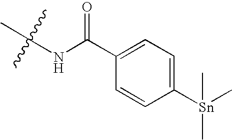 |
| 1039 | (S)-CN | 1 | 0 | 0 | 0 | — | 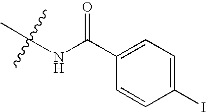 |
| 1040 | (S)-CN | 1 | 0 | 0 | 0 | — | 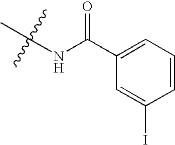 |
| 1041 | (S)-CN | 1 | 0 | 0 | 0 | — | 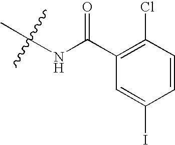 |
| 1042 | (S)-CN | 1 | 0 | 0 | 0 | — | 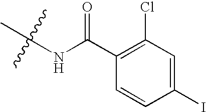 |
| 1043 | (S)-CN | 1 | 0 | 0 | 0 | — | 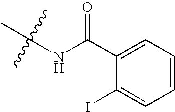 |
| 1044 | (S)-CN | 1 | 0 | 0 | 0 | — | 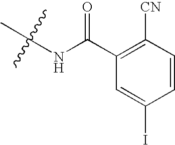 |
| 1045 | (S)-CN | 1 | 0 | 0 | 0 | — | 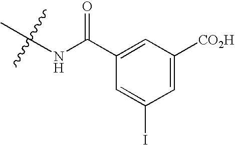 |
| 1046 | (R)-CN | 1 | 0 | 0 | 0 | — | 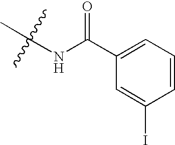 |
| 1047 | (S)-CN | 1 | 0 | 0 | 0 | — | 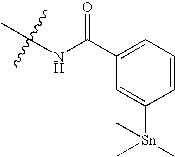 |
| 1048 | (S)-CN | 1 | 0 | 0 | 0 | — | 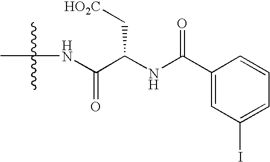 |
| 1049 | (S)-CN | 1 | 0 | 0 | 0 | — | 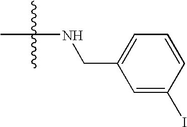 |
| 1050 | (S)-CN | 1 | 0 | 0 | 0 | — | 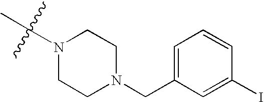 |
| 1060 | B(OH)₂ | 1 | 0 | 0 | 0 | — | 8M |
| | | | | | | | M = Re |
| 1070 | B(OH)₂ | 1 | 1 | 0 | 5 | — | 3 |
| 1071 | B(OH)₂ | 1 | 1 | 0 | 5 | — | 2M |
| | | | | | | | M = Re |
| 1072 | B(OH)₂ | 1 | 1 | 0 | 5 | — | 4M |
| | | | | | | | M = Re |
| 1073 | B(OH)₂ | 1 | 1 | 0 | 5 | — | 6M |
| | | | | | | | M = Re |
| 1074 | S—CN | 1 | 1 | 0 | 0 | — | 10 |
| 1075 | S—CN | 1 | 1 | 0 | 0 | — | 10M |
| | | | | | | | M = Re |
| 1076 | S—CN | 1 | 1 | 0 | 0 | — | 10M |
| | | | | | | | M = Tc |
| 1077 | R—CN | 1 | 1 | 0 | 5 | — | 10 |
| 1078 | R—CN | 1 | 1 | 0 | 5 | — | 10M |
| | | | | | | | M = Re |
| 1079 | R—CN | 1 | 1 | 0 | 5 | — | 10M |
| | | | | | | | M = Tc |
| 1080 | B(OH)₂ | 1 | 1 | 0 | 0 | — | 10 |
| 1081 | B(OH)₂ | 1 | 1 | 0 | 0 | — | 10M |
| | | | | | | | M = Re |
| 1082 | B(OH)₂ | 1 | 1 | 0 | 0 | — | 10M |
| | | | | | | | M = Tc |
| 1083 | B(OH)₂ | 1 | 1 | 0 | 5 | — | 10 |
| 1084 | B(OH)₂ | 1 | 1 | 0 | 5 | — | 10M |
| | | | | | | | M = Re |
| 1085 | B(OH)₂ | 1 | 1 | 0 | 5 | — | 10M |
| | | | | | | | M = Tc |
| 1086 | B(OH)₂ | 1 | 1 | 0 | 5 | — | 12M |
| | | | | | | | M = Re |
| 1087 | B(OH)₂ | 1 | 1 | 0 | 5 | — | 5M |
| | | | | | | | M = Re |
| 1088 | B(OH)₂ | 1 | 1 | 0 | 5 | — | 13M |
| | | | | | | | M = Re |
| 1089 | B(OH)₂ | 1 | 1 | 0 | 5 | — | 14M |
| | | | | | | | M = Re |
| 1090 | B(OH)₂ | 1 | 1 | 0 | 5 | — | 24M |
| | | | | | | | M = Re |
| 1091 | B(OH)₂ | 1 | 1 | 0 | 5 | — | 25M |
| | | | | | | | M = Re |
| 1092 | B(OH)₂ | 1 | 1 | 0 | 5 | — | 27 |
| 1093 | B(OH)₂ | 1 | 1 | 0 | 5 | — | 28 |
| 1094 | B(OH)₂ | 1 | 1 | 0 | 5 | — | 29 |
| | | | | | | | R = H, alkyl, or |
| | | | | | | | alkyloxyalkyl |
| 1095 | B(OH)₂ | 1 | 1 | 0 | 5 | — | 29M |
| | | | | | | | M = Re |
| | | | | | | | R = H, alkyl, or |
| | | | | | | | alkyloxyalkyl |
| 1096 | (S)-CN | 1 | 1 | 0 | 5 | — | 30 |
| 1097 | B(OH)₂ | 1 | 0 | 0 | 0 | — | 1M |
| | | | | | | | M = Re |
| 1098 | B(OH)₂ | 1 | 1 | 0 | 5 | — | 31M |
| | | | | | | | M = Tc |
| 1099 | B(OH)₂ | 1 | 0 | 0 | 0 | — | 32M |
| | | | | | | | M = Re |
| 1100 | B(OH)₂ | 1 | 1 | 0 | 5 | — | 32M |
| | | | | | | | M = Re |
| 1101 | B(OH)₂ | 1 | 1 | 0 | 5 | — | 33M |
| | | | | | | | M = Re |
| 1102 | B(OH)₂ | 1 | 0 | 0 | 0 | — | 9M |
| | | | | | | | M = Re |
| 1103 | B(OH)₂ | 1 | 0 | 0 | 0 | — | 3M |
| | | | | | | | M = Re |
| 1104 | B(OH)₂ | 5 | 0 | 0 | 0 | — | 1M |
| | | | | | | | M = Re |
| 1105 | B(OH)₂ | 1 | 1 | 1 | 4 | H | 32M |
| | | | | | | | M = Re |
| 1106 | B(OH)₂ | 1 | 1 | 0 | 4 | — | 9M |
| | | | | | | | M = Re |
| 1107 | B(OH)₂ | 1 | 1 | 0 | 4 | — | 34 |
| 1108 | B(OH)₂ | 1 | 1 | 0 | 4 | — | 35 |
| 1109 | B(OH)₂ | 1 | 1 | 0 | 5 | — | 1M |
| | | | | | | | M = Tc |
| 1110 | B(OH)₂ | 1 | 1 | 0 | 5 | — | 7M |
| | | | | | | | M = Tc |
| 1111 | B(OH)₂ | 1 | 1 | 0 | 5 | — | 11 |
| 1112 | B(OH)₂ | 1 | 1 | 0 | 5 | — | 15 |
| 1113 | B(OH)₂ | 1 | 1 | 0 | 5 | — | 16 |
| 1113 | B(OH)₂ | 1 | 1 | 0 | 5 | — | 17 |
| 1114 | B(OH)₂ | 1 | 1 | 0 | 5 | — | 18 |
| 1115 | B(OH)₂ | 1 | 1 | 0 | 5 | — | 20 |
| 1116 | B(OH)₂ | 1 | 1 | 0 | 5 | — | 21 |
| 1117 | B(OH)₂ | 1 | 1 | 0 | 5 | — | 22 |
| 1118 | B(OH)₂ | 1 | 1 | 0 | 5 | — | 23 |
| 1119 | B(OH)₂ | 1 | 1 | 0 | 5 | — | 26 |
| 1120 | B(OH)₂ | 1 | 1 | 0 | 5 | — | 30 |
| 1121 | B(OH)₂ | 1 | 1 | 0 | 5 | — | 31 |
| 1122 | B(OH)₂ | 1 | 1 | 0 | 5 | — | 33 |
| 1124 | B(OH)₂ | 1 | 1 | 0 | 5 | — | 36 |
| 1125 | CN | 1 | 1 | 0 | 5 | — | 15 |
| 1126 | CN | 1 | 1 | 0 | 5 | — | 16 |
| 1127 | CN | 1 | 1 | 0 | 5 | — | 17 |
| 1128 | CN | 1 | 1 | 0 | 5 | — | 18 |
| 1129 | CN | 1 | 1 | 0 | 5 | — | 20 |
| 1130 | CN | 1 | 1 | 0 | 5 | — | 21 |
| 1131 | CN | 1 | 1 | 0 | 5 | — | 22 |
| 1132 | CN | 1 | 1 | 0 | 5 | — | 23 |
| 1133 | CN | 1 | 1 | 0 | 5 | — | 26 |
| 1134 | CN | 1 | 1 | 0 | 5 | — | 30 |
| 1135 | CN | 1 | 1 | 0 | 5 | — | 31 |
| 1136 | CN | 1 | 1 | 0 | 5 | — | 33 |
| 1137 | CN | 1 | 1 | 0 | 5 | — | 33M |
| | | | | | | | M = Re |
| 1138 | CN | 1 | 1 | 0 | 5 | — | 36 |
| 1138 | B(OH)₂ | 1 | 1 | 0 | 5 | — | 9M |
| | | | | | | | M = Tc |
| 1140 | B(OH)₂ | 1 | 1 | 0 | 5 | — | 3M |
| | | | | | | | M = Tc |
| 1142 | B(OH)₂ | 1 | 1 | 0 | 5 | — | 8M |
| | | | | | | | M = Tc |
| 1143 | (S)-CN | 1 | 1 | 0 | 5 | — | 8M |
| | | | | | | | M = Tc |
| 1144 | B(OH)₂ | 1 | 0 | 0 | 0 | — | 8M |
| | | | | | | | M = Tc |
| 1145 | B(OH)₂ | 1 | 1 | 0 | 5 | — | 2M |
| | | | | | | | M = Tc |
| 1146 | B(OH)₂ | 1 | 1 | 0 | 5 | — | 4M |
| | | | | | | | M = Tc |
| 1147 | B(OH)₂ | 1 | 1 | 0 | 5 | — | 6M |
| | | | | | | | M = Tc |
| 1150 | B(OH)₂ | 1 | 1 | 0 | 5 | — | 12M |
| | | | | | | | M = Tc |
| 1151 | B(OH)₂ | 1 | 1 | 0 | 5 | — | 5M |
| | | | | | | | M = Tc |
| 1152 | B(OH)₂ | 1 | 1 | 0 | 5 | — | 13M |
| | | | | | | | M = Tc |
| 1153 | B(OH)₂ | 1 | 1 | 0 | 5 | — | 14M |
| | | | | | | | M = Tc |
| 1154 | B(OH)₂ | 1 | 1 | 0 | 5 | — | 24M |
| | | | | | | | M = Tc |
| 1155 | B(OH)₂ | 1 | 1 | 0 | 5 | — | 25M |
| | | | | | | | M = Tc |
| 1156 | B(OH)₂ | 1 | 1 | 0 | 5 | — | 29M |
| | | | | | | | M = Tc |
| | | | | | | | R = H, alkyl, or |
| | | | | | | | alkyloxyalkyl |
| 1157 | B(OH)₂ | 1 | 0 | 0 | 0 | — | 1M |
| | | | | | | | M = Tc |
| 1158 | B(OH)₂ | 1 | 0 | 0 | 0 | — | 32M |
| | | | | | | | M = Tc |
| 1159 | B(OH)₂ | 1 | 1 | 0 | 5 | — | 32M |
| | | | | | | | M = Tc |
| 1160 | B(OH)₂ | 1 | 1 | 0 | 5 | — | 33M |
| | | | | | | | M = Tc |
| 1161 | B(OH)₂ | 1 | 0 | 0 | 0 | — | 9M |
| | | | | | | | M = Tc |
| 1162 | B(OH)₂ | 1 | 0 | 0 | 0 | — | 3M |
| | | | | | | | M = Tc |
| 1163 | B(OH)₂ | 5 | 0 | 0 | 0 | — | 1M |
| | | | | | | | M = Tc |
| 1164 | B(OH)₂ | 1 | 1 | 1 | 4 | H | 32M |
| | | | | | | | M = Tc |
| 1165 | B(OH)₂ | 1 | 1 | 0 | 4 | — | 9M |
| | | | | | | | M = Tc |
| 1167 | CN | 1 | 1 | 0 | 5 | — | 33M |
| | | | | | | | M = Tc |
| 1168 | CN | 1 | 1 | 0 | 5 | — | 11 |
| 1169 | CN | 1 | 1 | 0 | 5 | — | 12M |
| | | | | | | | M = Re |
| 1170 | CN | 1 | 1 | 0 | 5 | — | 12M |
| | | | | | | | M = Tc |
| 1171 | CN | 1 | 1 | 0 | 5 | — | 13M |
| | | | | | | | M = Re |
| 1172 | CN | 1 | 1 | 0 | 5 | — | 13M |
| | | | | | | | M = Tc |
| 1173 | CN | 1 | 1 | 0 | 5 | — | 14M |
| | | | | | | | M = Re |
| 1174 | CN | 1 | 1 | 0 | 5 | — | 14M |
| | | | | | | | M = Tc |
| 1175 | CN | 1 | 0 | 0 | 4 | — | 1M |
| | | | | | | | M = Re |
| 1176 | CN | 1 | 1 | 0 | 5 | — | 1M |
| | | | | | | | M = Re |
| 1177 | CN | 1 | 0 | 0 | 0 | — | 1M |
| | | | | | | | M = Re |
| 1178 | CN | 5 | 0 | 0 | 0 | — | 1M |
| | | | | | | | M = Re |
| 1179 | CN | 1 | 1 | 0 | 5 | — | 1M |
| | | | | | | | M = Tc |
| 1180 | CN | 1 | 0 | 0 | 0 | — | 1M |
| | | | | | | | M = Tc |
| 1181 | CN | 5 | 0 | 0 | 0 | — | 1M |
| | | | | | | | M = Tc |
| 1182 | CN | 1 | 1 | 0 | 5 | — | 24M |
| | | | | | | | M = Re |
| 1183 | CN | 1 | 1 | 0 | 5 | — | 24M |
| | | | | | | | M = Tc |
| 1184 | CN | 1 | 1 | 0 | 5 | — | 25M |
| | | | | | | | M = Re |
| 1185 | CN | 1 | 1 | 0 | 5 | — | 25M |
| | | | | | | | M = Tc |
| 1186 | CN | 1 | 1 | 0 | 5 | — | 29M |
| | | | | | | | M = Re |
| | | | | | | | R = H, alkyl, or |
| | | | | | | | alkyloxyalkyl |
| 1187 | CN | 1 | 1 | 0 | 5 | — | 29M |
| | | | | | | | M = Tc |
| | | | | | | | R = H, alkyl, or |
| | | | | | | | alkyloxyalkyl |
| 1188 | CN | 1 | 1 | 0 | 5 | — | 2M |
| | | | | | | | M = Re |
| 1189 | CN | 1 | 1 | 0 | 5 | — | 2M |
| | | | | | | | M = Tc |
| 1190 | CN | 1 | 1 | 0 | 5 | — | 31M |
| | | | | | | | M = Tc |
| 1191 | CN | 1 | 0 | 0 | 0 | — | 32M |
| | | | | | | | M = Re |
| 1192 | CN | 1 | 1 | 0 | 5 | — | 32M |
| | | | | | | | M = Re |
| 1193 | CN | 1 | 1 | 1 | 4 | H | 32M |
| | | | | | | | M = Re |
| 1194 | CN | 1 | 0 | 0 | 0 | — | 32M |
| | | | | | | | M = Tc |
| 1195 | CN | 1 | 1 | 0 | 5 | — | 32M |
| | | | | | | | M = Tc |
| 1196 | CN | 1 | 1 | 1 | 4 | H | 32M |
| | | | | | | | M = Tc |
| 1197 | CN | 1 | 1 | 0 | 5 | — | 3M |
| | | | | | | | M = Re |
| 1198 | CN | 1 | 0 | 0 | 0 | — | 3M |
| | | | | | | | M = Re |
| 1199 | CN | 1 | 1 | 0 | 5 | — | 3M |
| | | | | | | | M = Tc |
| 1200 | CN | 1 | 0 | 0 | 0 | — | 3M |
| | | | | | | | M = Tc |
| 1201 | CN | 1 | 1 | 0 | 5 | — | 4M |
| | | | | | | | M = Re |
| 1202 | CN | 1 | 1 | 0 | 5 | — | 4M |
| | | | | | | | M = Tc |
| 1203 | CN | 1 | 1 | 0 | 5 | — | 5M |
| | | | | | | | M = Re |
| 1204 | CN | 1 | 1 | 0 | 5 | — | 5M |
| | | | | | | | M = Tc |
| 1205 | CN | 1 | 1 | 0 | 5 | — | 6M |
| | | | | | | | M = Re |
| 1206 | CN | 1 | 1 | 0 | 5 | — | 6M |
| | | | | | | | M = Tc |
| 1207 | CN | 1 | 1 | 0 | 5 | — | 7M |
| | | | | | | | M = Re |
| 1208 | CN | 1 | 1 | 0 | 5 | — | 7M |
| | | | | | | | M = Tc |
| 1209 | CN | 1 | 0 | 0 | 0 | — | 8M |
| | | | | | | | M = Re |
| 1210 | CN | 1 | 0 | 0 | 0 | — | 8M |
| | | | | | | | M = Tc |
| 1211 | CN | 1 | 1 | 0 | 5 | — | 9M |
| | | | | | | | M = Re |
| 1212 | CN | 1 | 0 | 0 | 0 | — | 9M |
| | | | | | | | M = Re |
| 1213 | CN | 1 | 1 | 0 | 4 | — | 9M |
| | | | | | | | M = Re |
| 1214 | CN | 1 | 1 | 0 | 5 | — | 9M |
| | | | | | | | M = Tc |
| 1215 | CN | 1 | 0 | 0 | 0 | — | 9M |
| | | | | | | | M = Tc |
| 1216 | CN | 1 | 1 | 0 | 4 | — | 9M |
| | | | | | | | M = Tc |

TABLE 4
Listing of Q variable groups.

| | Q |
|---|---|
| | Group |
| Q | Number |
| 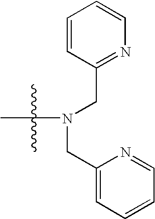 | 1 |
| 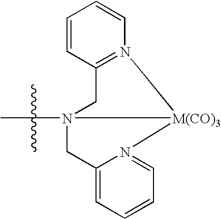 | 1M |
| 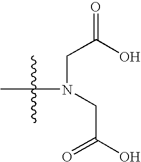 | 2 |
| 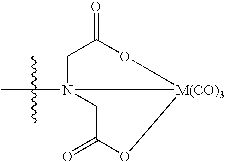 | 2M |
| 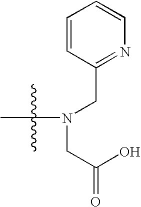 | 3 |
| 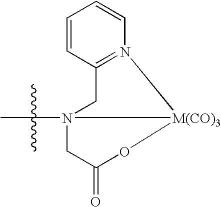 | 3M |
| 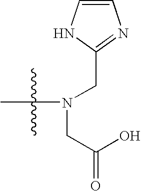 | 4 |
| 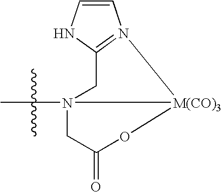 | 4M |
| 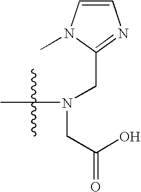 | 5 |
| 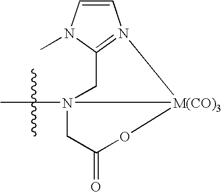 | 5M |
| 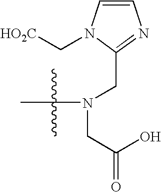 | 6 |
| 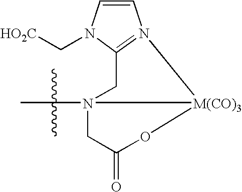 | 6M |
| 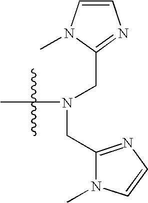 | 7 |
| 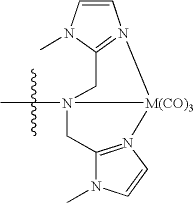 | 7M |
| 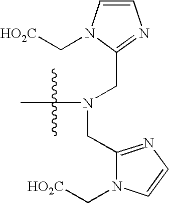 | 8 |
| 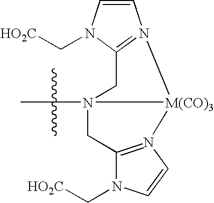 | 8M |
| 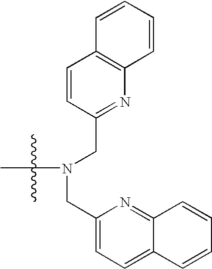 | 9 |
| 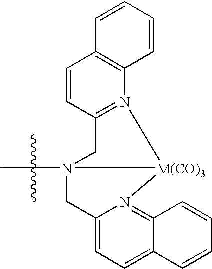 | 9M |
| 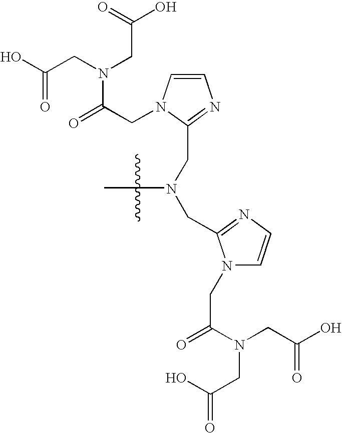 | 10 |
| 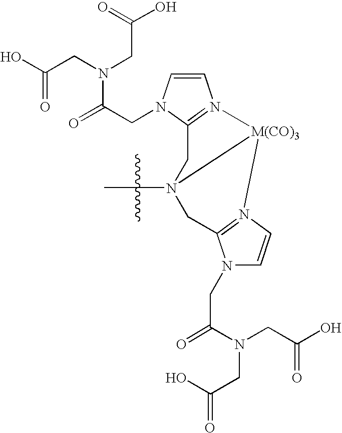 | 10M |
| 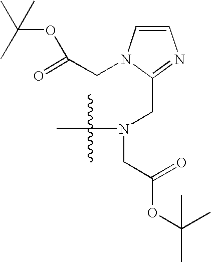 | 11 |
| 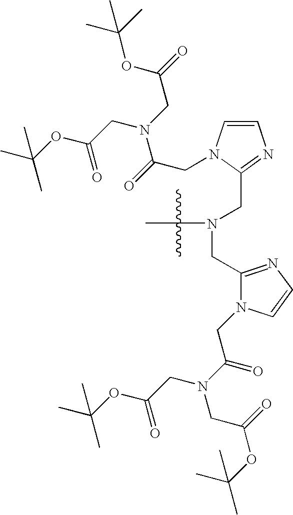 | 12 |
| 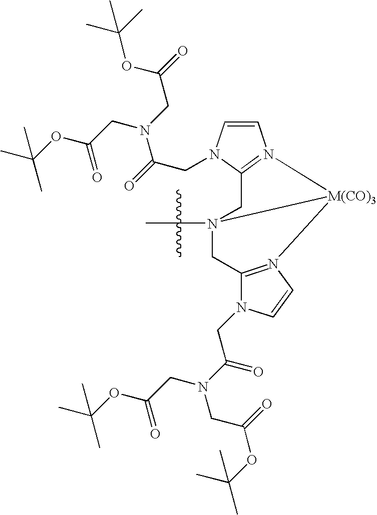 | 12M |
| 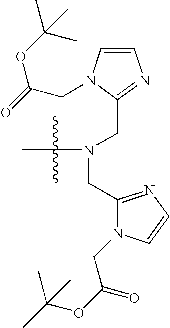 | 13 |
| 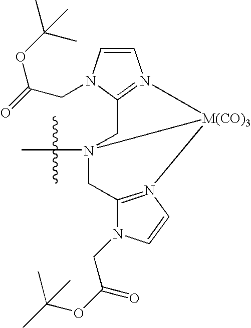 | 13M |
| 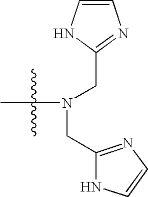 | 14 |
| 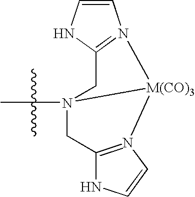 | 14M |
| 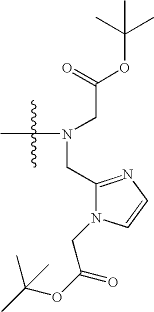 | 15 |
| 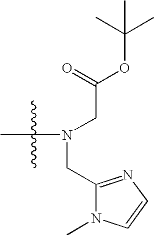 | 16 |
| 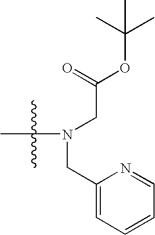 | 17 |
| 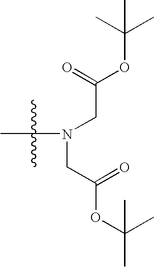 | 18 |
| 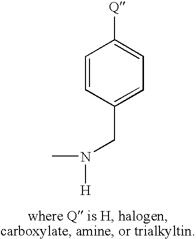 | 19 |
| 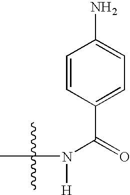 | 20 |
| 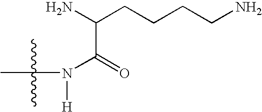 | 21 |
| 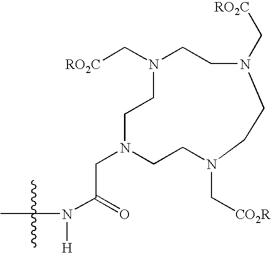 | 22 |
| 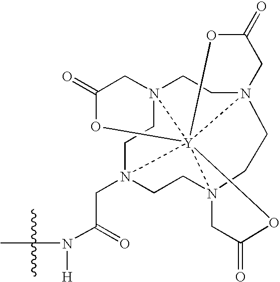 | 23 |
| 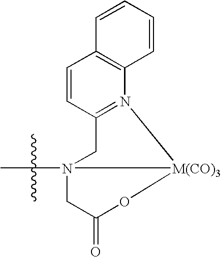 | 24M |
| 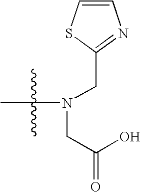 | 25 |
| 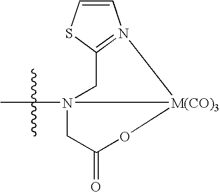 | 25M |
| 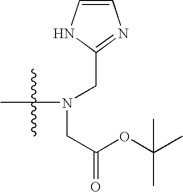 | 26 |
| 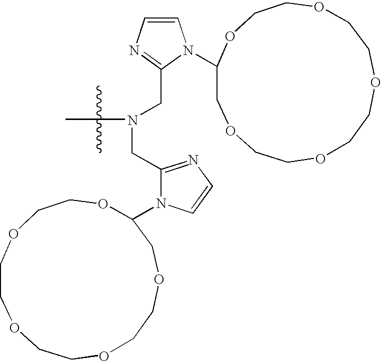 | 27 |
| 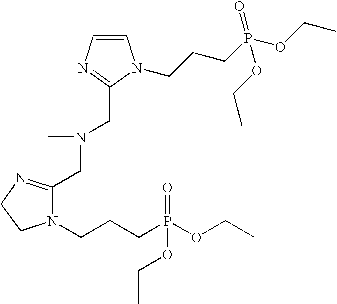 | 28 |
| 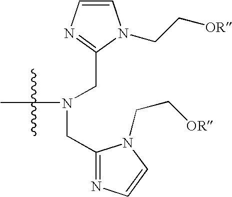 | 29 |
| 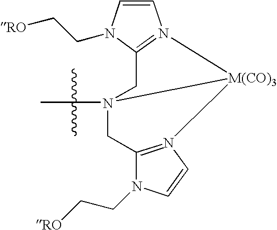 | 29M |
| 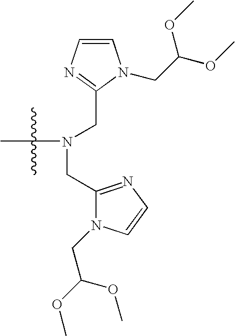 | 30 |
| 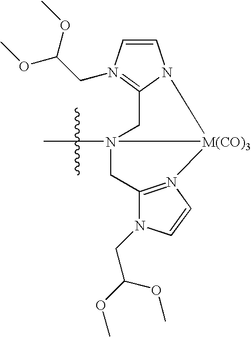 | 30M |
| 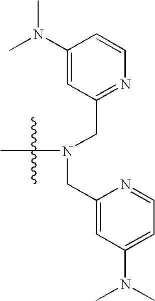 | 31 |
| 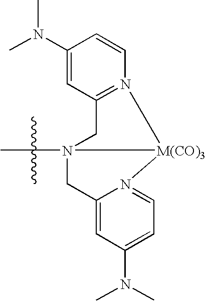 | 31M |
| 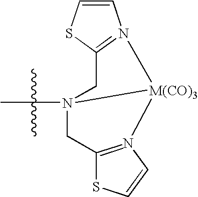 | 32M |
| 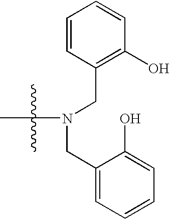 | 33 |
| 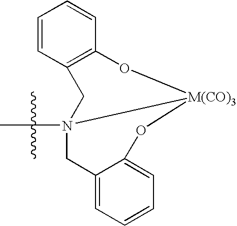 | 33M |
| 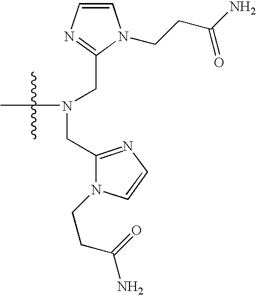 | 34 |
| 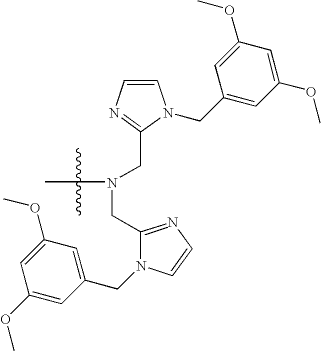 | 35 |
| 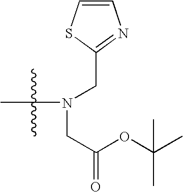 | 36 |

While certain embodiments have been illustrated and described, it should be understood that changes and modifications can be made therein in accordance with ordinary skill in the art without departing from the invention in its broader aspects as defined in the following claims.

What is claimed is:

1. A complex of Formula I, its stereoisomer or pharmaceutically acceptable salt: 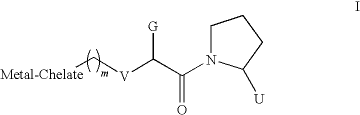
wherein:
U is selected from the group consisting of —B(OH)₂, —CN, —CO₂H and P(O)(OPh)₂;
G is selected from the group consisting of H, alkyl, substituted alkyl, carboxyalkyl, heteroalkyl, aryl, heteroaryl, heterocycle and arylalkyl;
V is a bond, O, S, NH, (CH₂—CH₂-X)ₙ or a group of 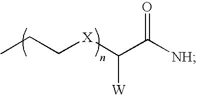
X is O, S, CH₂, or NR;
R is H, Me or CH₂CO₂H;
W is H or NHR′;
R′ is hydrogen, acetyl, t-butyloxycarbonyl (Boc), 9H-fluoren-9-ylmethoxycarbonyl (Fmoc), trifluoroacetyl, benzoyl, benzyloxycarbonyl (Cbz) or substituted benzoyl;
n is an integer ranging from 0 to 6;
m is an integer ranging from 0 to 6;
Metal represents a metallic moiety comprising a radionuclide; and
Chelate represents a chelating moiety that chelates to said Metal.

2. The complex of claim 1 wherein said radionuclide is selected from the group consisting of technetium-99m, technetium-94, rhenium-186, rhenium-188, lutetium-177, lutetium-170, yttrium-90, indium-111, gallium-67, gallium-68, copper-62, copper-64, copper-67, Bismuth-212, Astatine-211, Strontium-89, Holmium-166, Samarium-153, Palladium-100, Palladium-109, Lead-212, Rhodium-105 and Ruthenium-95.

3. The complex of claim 1 which has the structure of Formula I-a: 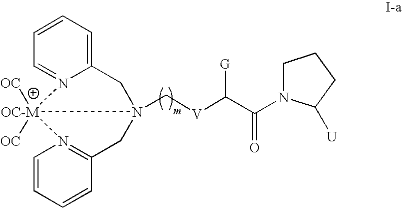
wherein:
M is technetium-99m (^{99m}Tc), rhenium-186 (¹⁸⁶Re) rhenium-188 (¹⁸⁸Re).

4. The complex of claim 1 which has the structure of Formula I-b: 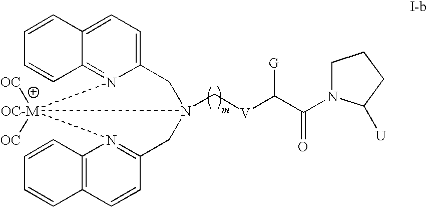
wherein:
M is said Metal and is selected from the group consisting of technetium-99m (^{99m}Tc), rhenium-186 (¹⁸⁶Re) and rhenium-188 (¹⁸⁸Re).

5. The complex of claim 1 which has the structure of Formula I-c: 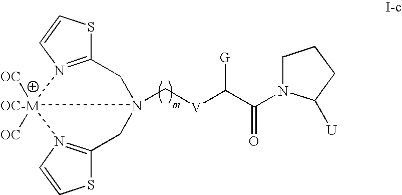
wherein:
M is said Metal and is selected from the group consisting of technetium-99m (^{99m}Tc), rhenium-186 (¹⁸⁶Re) and rhenium-188 (¹⁸⁸Re).

6. The complex of claim 1 which has the structure of Formula I-d: 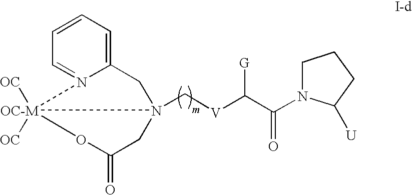
wherein:
M is said Metal and is selected from the group consisting of technetium-99m (^{99m}Tc), rhenium-186 (¹⁸⁶Re) and rhenium-188 (¹⁸⁸Re).

7. The complex of claim 1 which has the structure of Formula I-e: 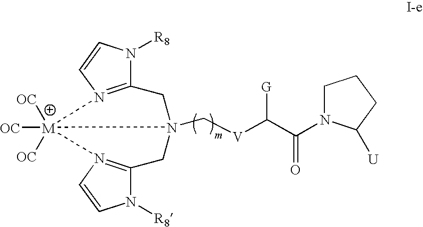
wherein:
R₈ and R₈′ are each independently hydrogen, halogen, a substituted or unsubstituted alkyl, alkenyl, alkynyl, hydroxyl, alkoxyl, acyl, acyloxy, acylamino, silyloxy, amino, monoalkylamino, dialkylamino, nitro, sulfhydryl, alkylthio, imino, amido, phosphoryl, phosphonate, phosphine, carbonyl, carboxyl, carboxamide, anhydride, silyl, thioalkyl, alkylsulfonyl, arylsulfonyl, selenoalkyl, ketone, aldehyde, ether, ester, heteroalkyl, cyano, guanidine, amidine, acetal, ketal, amine oxide, aryl, heteroaryl, aralkyl, arylether, heteroaralkyl, azido, aziridine, carbamoyl, epoxide, hydroxamic acid, imide, oxime, sulfonamide, thioamide, thiocarbamate, urea, thiourea, (CH₂)_{d}CO₂H, CH₂CH₂OCH₂CH₃, CH₂CH(OCH₃)₂, (CH₂CH₂O)_{d}CH₂CH₃, (CH₂)_{d}C(O)N((CH₂)_{d}COOH)₂, (CH₂)_{d}NH₂, CH₂CH₂C(O)NH₂, (CH₂)_{d}N(CH₃)₂, CH₂CH₂OH, (CH₂)_{d}CH(CO₂H)₂, (CH₂)_{d}P(O)(OH)₂, (CH₂)_{d}B(OH)₂, or —(CH₂)_{d}—R₉;
each d is individually an integer from 0 to 6;
each R₉ is independently 15-Crown-5,18-Crown-6, tetrazole, oxazole, aziridine, triazole, imidazole, pyrazole, thiazole, hydroxamic acid, phosphonate, phosphinate, thiol, thioether, polysachamide, sachamide, nucleotide or oligonucleotide; and
M is said Metal and is selected from the group consisting of technetium-99m (^{99m}Tc), rhenium-186 (¹⁸⁶Re) and rhenium-188 (¹⁸⁸Re).

8. The complex of claim 7, wherein R₈ and R₈′ are CH₂C(O)N(CH₂COOH)₂.

9. The complex of claim 7, wherein R₈ and R₈′ are CH₂COOH.

10. The complex of claim 1 which has the structure of Formula I-f: 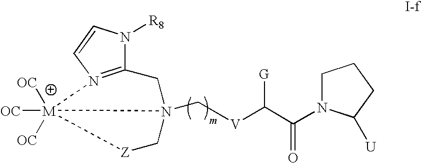
wherein:
Z is a substituted or unsubstituted thioalkyl, carboxylate, carboxyalkyl, aminoalkyl, heterocyclyl, (amino acid), (amino acid)alkyl, hydroxy, hydroxyalkyl, 2-(carboxy)aryl, 2-(carboxy)heteroaryl, 2-(hydroxy)aryl, 2-(hydroxy)heteroaryl, 2-(thiol)aryl, 2-pyrrolidine boronic acid, or 2-(thiol)heteroaryl;
R₈ is independently hydrogen, halogen, a substituted or unsubstituted alkyl, alkenyl, alkynyl, hydroxyl, alkoxyl, acyl, acyloxy, acylamino, silyloxy, amino, monoalkylamino, dialkylamino, nitro, sulfhydryl, alkylthio, imino, amido, phosphoryl, phosphonate, phosphine, carbonyl, carboxyl, carboxamide, anhydride, silyl, thioalkyl, alkylsulfonyl, arylsulfonyl, selenoalkyl, ketone, aldehyde, ether, ester, heteroalkyl, cyano, guanidine, amidine, acetal, ketal, amine oxide, aryl, heteroaryl, aralkyl, arylether, heteroaralkyl, azido, aziridine, carbamoyl, epoxide, hydroxamic acid, imide, oxime, sulfonamide, thioamide, thiocarbamate, urea, thiourea, (CH₂)_{d}CO₂H, CH₂CH₂OCH₂CH₃, CH₂CH(OCH₃)₂, (CH₂CH₂O)_{d}CH₂CH₃, (CH₂)_{d}C(O)N((CH₂)_{d}COOH)₂, (CH₂)_{d}NH₂, CH₂CH₂C(O)NH₂, (CH₂)_{d}N(CH₃)₂, CH₂CH₂OH, (CH₂)_{d}CH(CO₂H)₂, (CH₂)_{d}P(O)(OH)₂, (CH₂)_{d}B(OH)₂, or —(CH₂)_{d}—R₉;
each d is individually an integer from 0 to 6;
each R₉ is independently 15-Crown-5,18-Crown-6, tetrazole, oxazole, aziridine, triazole, imidazole, pyrazole, thiazole, hydroxamic acid, phosphonate, phosphinate, thiol, thioether, polysachamide, sachamide, nucleotide or oligonucleotide; and
M is said Metal and is selected from the group consisting of technetium-99m (^{99m}Tc), rhenium-186 (¹⁸⁶Re) and rhenium-188 (¹⁸⁸Re).

11. The complex of claim 1 which has the structure of Formula I-g: 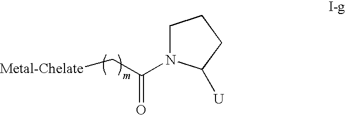
wherein:
said radionuclide is selected from the group consisting of technetium-99m (^{99m}Tc), rhenium-186 (¹⁸⁶Re).

12. The complex of claim 1 which has the structure of Formula I-h: 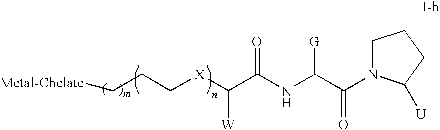
wherein:
said radionuclide is selected from the group consisting of technetium-99m (^{99m}Tc), rhenium-186 (¹⁸⁶Re) and rhenium-188 (¹⁸⁸Re).

13. The complex of claim 1 which has the structure of Formula I-i: 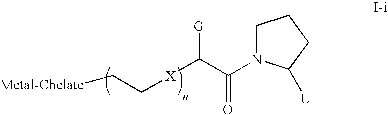
wherein:
said radionuclide is selected from the group consisting of technetium-99m (^{99m}Tc), rhenium-186 (¹⁸⁶Re) and rhenium-188 (¹⁸⁸Re).

14. The complex of claim 1, wherein said Chelate is selected from the group consisting of tetra-azacyclododecanetetra-acetic acid, diethylenetriaminepentaacetic acid bis(pyridin-2-ylmethyl)amine, quinolinemethylamino acetic acid, 2,2′-azanediyldiacetic acid, 2,2′-azanediylbis(methylene)diphenol, 2-((1H-imidazol-2-yl)methylamino)acetic acid, bis(isoquinolinemethyl)amine, bis(quinolinemethyl)amine, pyridine-2-ylmethylamino acetic acid, 2-(isoquinolin-3-ylmethylamino)acetic acid, bis((1H-imidazol-2-yl)methyl)amine, bis(thiazol-2-ylmethyl)amine, 2-(thiazol-2-ylmethylamino)acetic acid, 2,2′-(2,2′-azanediylbis(methylene)bis(1H-imidazole-2,1-diyl)diacetic acid, 2-((1-(carboxymethyl)-1H-imidazol-2-yl)methylamino)acetic acid, 2,2′-(2-(2-(azanediylbis(methyl ene)bis(1H-imidazol-1-yl)acetylazanediyl)diacetic acid and his (5-dimethylamino pyridine-2-ylmethyl)amine.

15. The complex of claim 1 wherein said radionuclide is gamma, positron or beta emitting.

16. A compound of general Formula II, its stereoisomer or pharmaceutically acceptable salt: 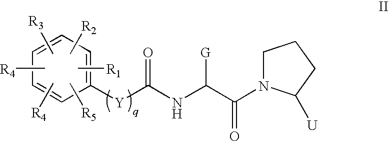
wherein:
U is selected from the group consisting of —B(OH)₂, —CN, —CO₂H and —P(O)(OPh)₂;
G is selected from the group consisting of H, alkyl, substituted alkyl, carboxyalkyl, heteroalkyl, aryl, heteroaryl, heterocycle and arylalkyl;
Y is a bond, —O—, —CH₂—, —OCH₂—, —CH₂O—, NR, —NR—CH₂, or CH₂—NR—, wherein R is H, Me or CH₂CO₂H;
q is an integer ranging from 0 to 24; and
R₁, R₂, R₃, R₄ and R₅ are independently selected from the group consisting of hydrogen, halogen, cyano, carboxyl, alkyl, alkylamino, alkoxy, and substituted or unsubstituted amino, provided that at least one of R₁, R₂, R₃, R₄ and R₅ is a radiohalogen.

17. The compound of claim 16, wherein said radiohalogen is selected from the group consisting of radioiodine and radiofluorine.

18. The compound of claim 16, which has the structure of Formula II-a: 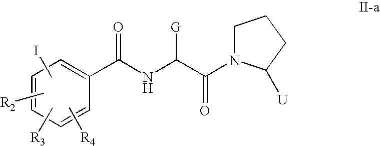
wherein:
R₂, R₃, and R₄ are independently selected from the group consisting of hydrogen, halogen, cyano, carboxyl, alkyl, alkylamino, alkoxy, and substituted or unsubstituted amino; and
I is radioiodine.

19. The compound of claim 16 which has the structure of Formula II-b: 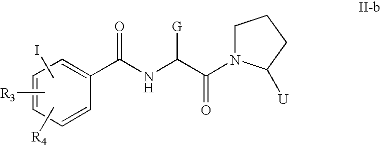
wherein:
R₃, and R₄ are independently selected from the group consisting of hydrogen, halogen, cyano, carboxyl, alkyl, alkylamino, alkoxy, and substituted or unsubstituted amino; and
I is radioiodine.

20. The compound of claim 16 which has the structure of Formula II-c: 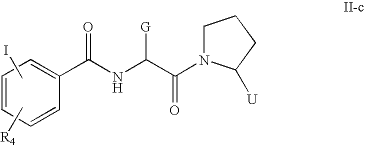
wherein:
R₄ is selected from the group consisting of hydrogen, halogen, cyano, carboxyl, alkyl, alkylamino, alkoxy, and substituted or unsubstituted amino; and
I is radioiodine.

21. The compound of claim 16 which has the structure of Formula II-d: 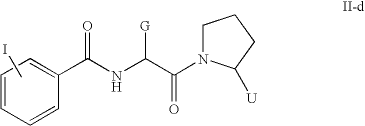
wherein:
I is radioiodine.

22. A method of imaging tissue of a mammal which expresses seprase comprising administering to said mammal an effective amount of a complex or compound, its enantiomer, stereoisomer, racemate or pharmaceutically acceptable salt, the complex or compound selected from the group consisting of formulae I and II: 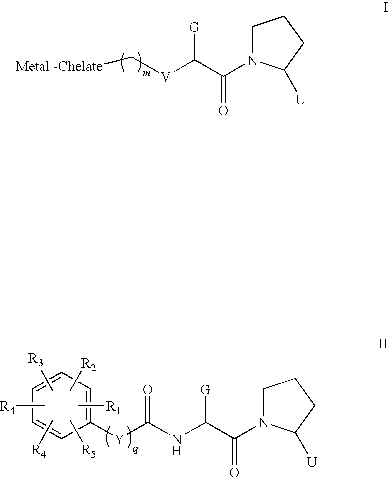
wherein:
U is selected from the group consisting of —B(OH)₂, —CN, —CO₂H and —P(O)(OPh)₂;
G is selected from the group consisting of H, alkyl, substituted alkyl, carboxyalkyl, heteroalkyl, aryl, heteroaryl, heterocycle and arylalkyl;
V is a bond, O, S, NH, (CH₂—CH₉—X) or a group of 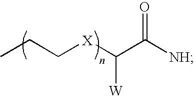
X is O, S, CH₂, or NR;
R is H, Me or CH₂CO₂H;
W is H or NHR′;
R′ is hydrogen, acetyl, t-butyloxycarbonyl (Boc), 9H-fluoren-9-ylmethoxycarbonyl (Fmoc), trifluoroacetyl, benzoyl, benzyloxycarbonyl (Cbz) or substituted benzoyl;
R₁, R₂, R₃, R₄ and R₅ are independently selected from the group consisting of hydrogen, halogen, cyano, carboxyl, alkyl, alkylamino, alkoxy, and substituted or unsubstituted amino provided that at least one of R₁, R₂, R₃, R₄ and R₅ is a radiohalogen;
Y is a bond, —O—, —CH₂—, —OCH₂—, —CH₂O—, NR, —NR—CH₂ or CH₂—NR—;
n is an integer ranging from 0 to 6;
m is an integer ranging from 0 to 6;
q is an integer ranging from 0 to 24;
Metal represents a metallic moiety comprising a radionuclide; and
Chelate represents a chelating moiety that chelates to said Metal;

23. The method of claim 22 wherein said complex is selected from the group consisting of 1-a to I-i: 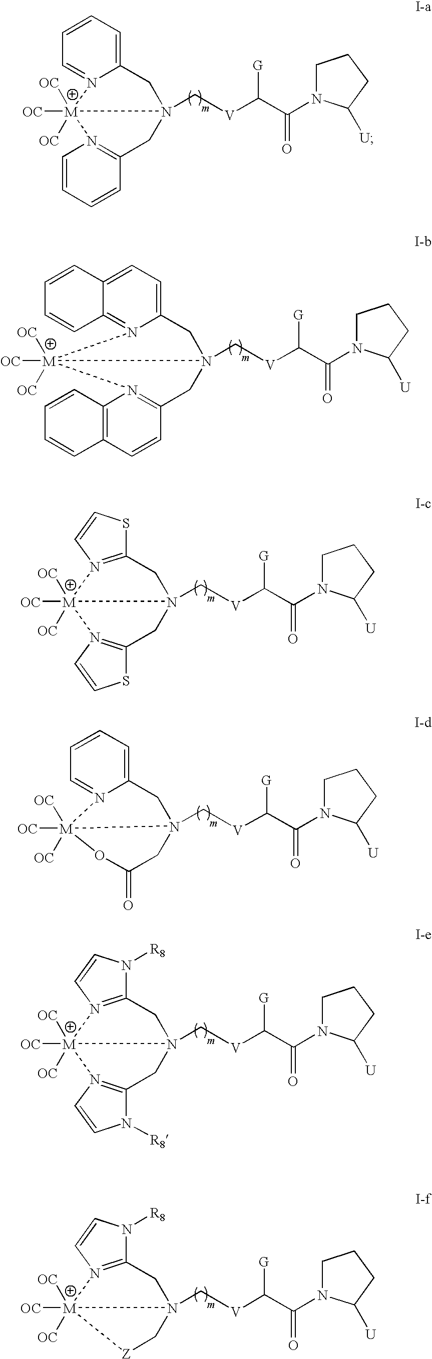 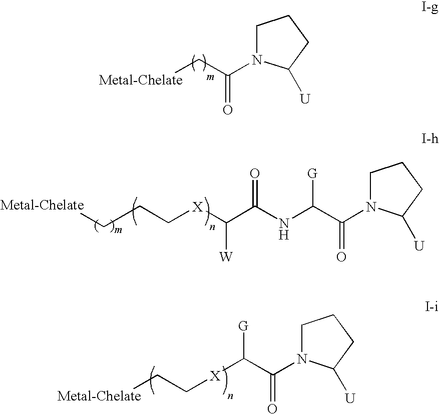
wherein:
Z is a substituted or unsubstituted thioalkyl, carboxylate, carboxyalkyl, aminoalkyl, heterocyclyl, (amino acid), (amino acid)alkyl, hydroxy, hydroxyalkyl, 2-(carboxy)aryl, 2-(carboxy)heteroaryl, 2-(hydroxy)aryl, 2-(hydroxy)heteroaryl, 2-(thiol)aryl, 2-pyrrolidine boronic acid, or 2-(thiol)heteroaryl;
R₈ and R₈′ are independently hydrogen, halogen, a substituted or unsubstituted alkyl, alkenyl, alkynyl, hydroxyl, alkoxyl, acyl, acyloxy, acylamino, silyloxy, amino, monoalkylamino, dialkylamino, nitro, sulfhydryl, alkylthio, imino, amido, phosphoryl, phosphonate, phosphine, carbonyl, carboxyl, carboxamide, anhydride, silyl, thioalkyl, alkylsulfonyl, arylsulfonyl, selenoalkyl, ketone, aldehyde, ether, ester, heteroalkyl, cyano, guanidine, amidine, acetal, ketal, amine oxide, aryl, heteroaryl, aralkyl, arylether, heteroaralkyl, azido, aziridine, carbamoyl, epoxide, hydroxamic acid, imide, oxime, sulfonamide, thioamide, thiocarbamate, urea, thiourea, (CH₂)_{d}CO₂H, CH₂CH₂OCH₂CH₃, CH₂CH(OCH₃)₂, (CH₂CH₂O)_{d}CH₂CH₃, (CH₂)_{d}NH₂, CH₂CH₂C(O)NH₂, (CH₂)_{d}N(CH₃)₂, CH₂CH₂OH, (CH₂)_{d}CH(CO₂H)₂, (CH₂)_{d}P(O)(OH)₂, (CH₂)_{d}B(OH)₂, or —(CH₂)_{d}—R₉, wherein d is an integer from 0 to 6 and R₉ is each independently 15-Crown-5, 18-Crown-6, tetrazole, oxazole, aziridine, triazole, imidazole, pyrazole, thiazole, hydroxamic acid, phosphonate, phosphinate, thiol, thioether, polysachamide, sachamide, nucleotide or oligonucleotide; and
M is technetium-99m (^{99m}Tc), rhenium-186 (¹⁸⁶Re) or rhenium-188 (¹⁸⁸Re).

24. The method of claim 22 wherein said compound is selected from the group consisting of Formula II-a, II-b, II-c, and II-d: 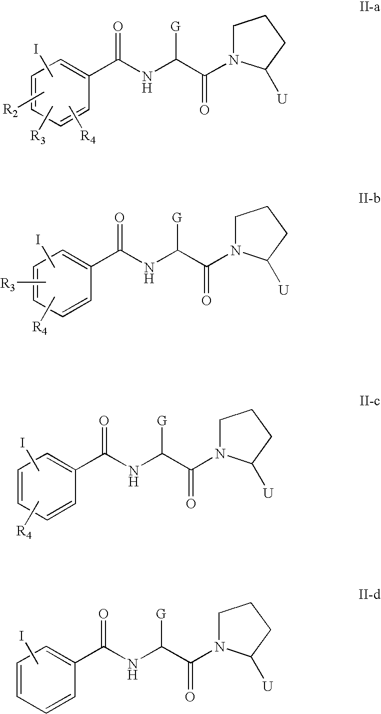
wherein
R₂, R₃, and R₄ are independently selected from the group consisting of hydrogen, halogen, cyano, carboxyl, alkyl, alkylamino, alkoxy, and substituted or unsubstituted amino; and
I is radioiodine.

25. A method of treating a mammal suffering a disease which is characterized by overexpression of seprase, the method comprising administering to said mammal a therapeutically effective amount of a complex or compound, its stereoisomer or pharmaceutically acceptable salt, selected from the group consisting of formulae I and II: 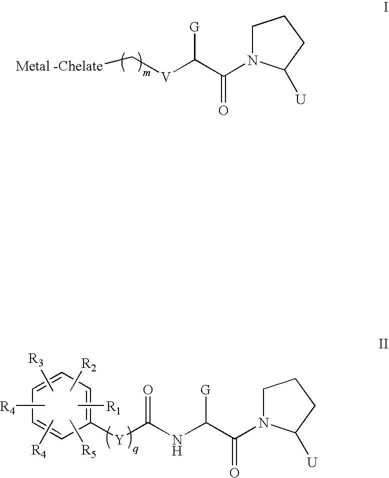
wherein:
U is selected from the group consisting of —B(OH)₂, —CN, —CO₂H and —P(O)(OPh)₂;
G is selected from the group consisting of H, alkyl, substituted alkyl, carboxyalkyl, heteroalkyl, aryl, heteroaryl, heterocycle and arylalkyl;
V is a bond, O, S, NH, (CH₂—CH₂—X)ₙ or a group of 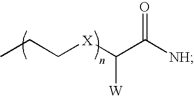
X is O, S, CH₂, or NR;
R is H, Me or CH₂CO₂H;
W is H or NHR′;
R′ is hydrogen, acetyl, t-butyloxycarbonyl (Boc), 9H-fluoren-9-ylmethoxycarbonyl (Fmoc), trifluoroacetyl, benzoyl, benzyloxycarbonyl (Cbz) or substituted benzoyl;
R₁, R₂, R₃, R₄ and R₅ are independently selected from the group consisting of hydrogen, halogen, cyano, carboxyl, alkyl, alkylamino, alkoxy, and substituted or unsubstituted amino provided that at least one of R₁, R₂, R₃, R₄ and R₅ is a radiohalogen;
Y is a bond, —O—, —CH₂—, —OCH₂—, —CH₂O—, NR, —NR—CH₂ or CH₂—NR—;
n is an integer ranging from 0 to 6;
m is an integer ranging from 0 to 6;
q is an integer ranging from 0 to 24;
Metal represents a metallic moiety comprising a radionuclide; and
Chelate represents a chelating moiety that chelates to said Metal;

26. The method of claim 25 wherein said complex is selected from the group consisting of I-a to I-i: 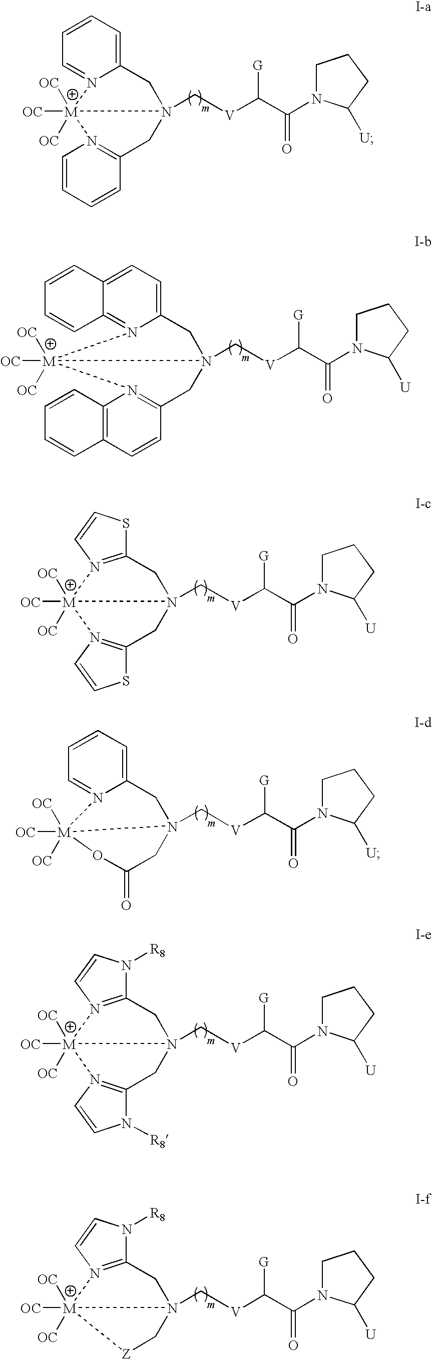 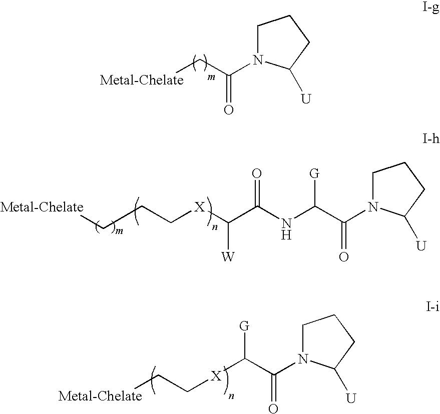
wherein:
Z is a substituted or unsubstituted thioalkyl, carboxylate, carboxyalkyl, aminoalkyl, heterocyclyl, (amino acid), (amino acid)alkyl, hydroxy, hydroxyalkyl, 2-(carboxy)aryl, 2-(carboxy)heteroaryl, 2-(hydroxy)aryl, 2-(hydroxy)heteroaryl, 2-(thiol)aryl, 2-pyrrolidine boronic acid, or 2-(thiol)heteroaryl;
R₈ and R₈′ are independently hydrogen, halogen, a substituted or unsubstituted alkyl, alkenyl, alkynyl, hydroxyl, alkoxyl, acyl, acyloxy, acylamino, silyloxy, amino, monoalkylamino, dialkylamino, nitro, sulfhydryl, alkylthio, imino, amino, phosphoryl, phosphonate, phosphine, carbonyl, carboxyl, carboxamide, anhydride, silyl, thioalkyl, alkylsulfonyl, arylsulfonyl, selenoalkyl, ketone, aldehyde, ether, ester, heteroalkyl, cyano, guanidine, amidine, acetal, ketal, amine oxide, aryl, heteroaryl, aralkyl, arylether, hetero aralkyl, azido, aziridine, carbamoyl, epoxide, hydroxamic acid, imide, oxime, sulfonamide, thioamide, thiocarbamate, urea, thiourea, (CH₂)_{d}CO₂H, CH₂CH₂OCH₂CH₃, CH₂CH(OCH₃)₂, (CH₂CH₂O)_{d}CH₂CH₃, (CH₂)_{d}C(O)N((CH₂)_{d}COOH)₂, (CH₂)_{d}NH₂, CH₂CH₂C(O)NH₂, (CH₂)_{d}N(CH₃)₂, CH₂CH₂OH, (CH₂)_{d}CH(CO₂H)₂, (CH₂)_{d}P(O)(OH)₂, (CH₂)_{d}B(OH)₂, or —(CH₂)_{d}—R₉;
each d is individually an integer from 0 to 6;
each R₉ is independently 15-Crown-5,18-Crown-6, tetrazole, oxazole, aziridine, triazole, imidazole, pyrazole, thiazole, hydroxamic acid, phosphonate, phosphinate, thiol, thioether, polysachamide, sacharride, nucleotide or oligonucleotide; and
M is technetium-99m (^{99m}Tc), rhenium 186 (¹⁸⁶Re),

27. The method of claim 25 wherein said compound is selected from the group consisting of Formula II-a, II-b, II-c, and II-d: 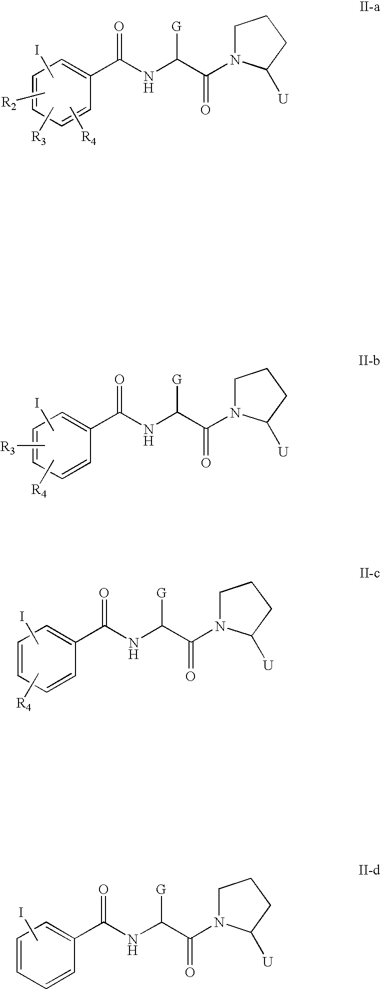
wherein
R₂, R₃, and R₄ are independently selected from the group consisting of hydrogen, halogen, cyano, carboxyl, alkyl, alkylamino, alkoxy, and substituted or unsubstituted amino; and
I is radioiodine.

28. A method of imaging tissue of a mammal which expresses seprase comprising administering to said mammal an effective amount of a radiolabeled seprase inhibitor.

29. A method of treating a mammal suffering from cancer comprising administering to said mammal an effective amount of a compound comprising a seprase inhibitor that is labeled with a therapeutic radionuclide wherein said radionuclide comprise a chelated metal or a halide.

30. The method of claim 25 in which said disease is cancer.
